(19) United States
(12) Patent Application Publication
(10) Pub. No.: US 2012/0101039 A1
(43) Pub. Date: Apr. 26, 2012

(54) CALCIUM-SENSING RECEPTOR-ACTIVE COMPOUNDS

(75) Inventors: Jef Fenscholdt, Stenlose (DK); Thomas Høyer, Charlottenlund (DK); Xifu Liang, Glostrup (DK); Sophie Elisabeth Havez, Valby (DK)

(73) Assignee: LEO PHARMA A/S, Ballerup (DK)

(21) Appl. No.: 13/322,189

(22) PCT Filed: May 26, 2010
(86) PCT No.: PCT/DK2010/000069

§ 371 Date: Jan. 12, 2012

Related U.S. Application Data
(60) Provisional application No. 61/181,566, filed on May 27, 2009.

(30) Foreign Application Priority Data
May 27, 2009 (DK) PA 2009 00663

(51) Int. Cl. A61K 38/23; A61K 31/24; C07C 63/36; A61K 31/195; C07D 295/10; A61K 31/5375; C07D 211/62; A61K 31/445; C07D 315/00; A61K 31/351; A61K 31/59; A61P 25/28; A61P 3/00; A61P 19/00; A61P 5/00; A61P 19/10; A61P 1/00; C07C 69/76

(52) U.S. Cl. 514/11.9; 560/100; 514/539; 562/490; 514/567; 544/162; 514/238.8; 546/245; 514/315; 549/425; 514/451; 514/167

(57) ABSTRACT

A compound of general formula I their use as calcium receptor-active compounds for the prophylaxis, treatment or amelioration of physiological disorders or diseases associated with disturbances of CaSR activity, such as hyperparathyroidism, pharmaceutical compositions comprising said compounds, methods of treating diseases with said compounds, and the use of said compounds in the manufacture of medicaments.

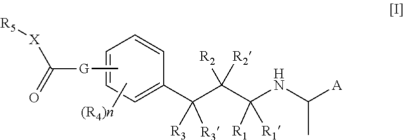

### FIELD OF THE INVENTION

This invention relates to novel calcium-sensing receptor-active compounds, to said compounds for use in therapy, to pharmaceutical compositions comprising said compounds, to methods of treating diseases with said compounds, and to the use of said compounds in the manufacture of medicaments.

### BACKGROUND OF THE INVENTION

The calcium-sensing receptor (CaSR) is a G-protein-coupled receptor (GPCR) that signals through the activation of phospholipase C, increasing levels of inositol 1,4,5-triphosphate and cytosolic calcium. The CaSR belongs to the subfamily C of the GPCR superfamily, which also includes receptors for glutamate, gamma aminobutyric acid (GABA), pheromones and odorants that all possess a very large extra-cellular domain. This domain is highly negatively charged and is involved in binding of calcium and other positively charged molecules. The CaSR is found in the parathyroid glands but has also been identified in the brain, intestine, pituitary, thyroid glands, bone tissue and kidneys [Brown, E. M. Calcium-Sensing Receptor. Primer of the Metabolic Bone Diseases and Disorders of Mineral Metabolism Fifth Edition, 2003 by American Society for Bone and Mineral Research, Chapter 17, p. 111.; Drueke, T. E. Nephrol Dial Transplant (2004) 19, v20-v26].

The calcium sensing receptor (CaSR) detects changes in extra-cellular calcium concentration and initiates the functional response of this cell, which is a modulation of the secretion of the parathyroid hormone (PTH). Secretion of PTH increases extra-cellular calcium ion concentration by acting on various cells, such as bone and kidney cells, and the extra-cellular calcium ion concentration reciprocally inhibits the secretion of PTH by acting on parathyroid cells. The reciprocal relationship between calcium concentration and PTH level is an essential mechanism for calcium homeostasis maintenance.

The calcimimetic activity corresponds to the ability to produce or induce biological responses observed through variations in the concentration of extracellular calcium ions (Ca²⁺)ₑ and extracellular magnesium ions (Mg²⁺)ₑ.

(Ca²⁺)ₑ and (Mg²⁺)ₑ ions play a major role in the body since they regulate calcium homeostasis on which the vital functions of the body depend. Thus, hypo- and hypercalcemia, that is to say conditions in which (Ca²⁺)ₑ ions are below or above the mean threshold, have a major effect on many functions, such as cardiac, renal or intestinal functions. They deeply affect the central nervous system (Chattopadhyay et al. Endocr. Review, 1996).

It has been shown that Ca²⁺ and Mg²⁺ ions, but also Ba²⁺ ions, within millimolar concentration ranges, stimulate CaSRs. Activation of CaSRs might be induced in the brain by β-amyloid peptides, which are involved in neurodegenerative diseases such as Alzheimer's disease (Ye et al, J. Neurosci., 47, 547-554, Res. 1997).

Disturbance of CaSR activity is associated with biological disorders such as primary and secondary hyperparathyroidism, osteoporosis, cardiovascular, gastrointestinal, endocrine and neurodegenerative diseases, or certain cancers in which (Ca²⁺)ₑ ions are abnormally high.

Primary hyperparathyroidism (primary HPT) is characterised by elevated levels of PTH and serum calcium which is typically caused by adenoma of the parathyroid gland. It can result in bone pain and excessive bone resorption.

Secondary hyperparathyroidism (secondary HPT) often develops in patients who have reduced kidney function and is characterised by elevated levels of PTH. The underlying causes are complex, but a reduced ability to convert vitamin D to calcitriol and elevated levels of phosphorus play significant roles in the development of secondary HPT. If left untreated, the clinical manifestations of secondary HPT include bone and joint pain and limb deformities [Harrington, P. E. and Fotsch, C. Calcium Sensing Receptor Activators: Calcimimetics. Current Medicinal Chemistry, 2007, 14, 3027-3034].

A reduced kidney function or renal failure is also accompanied by renal osteodystrophy, e.g. osteitis fibrosa, osteomalacia, adynamic bone disease, or osteoporosis. The disorders are characterized by either high or low bone turnover. Osteoporosis is a multifactor disease which depends in particular on age and sex. While menopausal women are very greatly affected, osteoporosis is increasingly proving to be a problem in elderly men, and, for the moment, no really satisfactory treatments exist. Its social cost may become even heavier in the years to come, particularly as life expectancy is becoming longer. Osteoporosis is currently treated with estrogens, calcitonin or biphosphonates which prevent bone resorption without stimulating bone growth. More recent data demonstrate that intermittent increases in PTH or in derivatives thereof are effective in the treatment of osteoporosis and make it possible to remodel bone by stimulating bone formation (Whitfield et al., 1999). This new therapeutic approach for treatment of osteoporosis appears to be very advantageous, although major problems are associated with the use of PTH hormone, such as the route of injection, but also the appearance of tumors, observed recently during clinical trials in humans. Intermittent secretion of endogenous PTH can be obtained by blocking the calcium sensing receptor. The blocking of PTH secretion with CaSR agonists may be followed by a rapid increase in PTH (rebound effect), which is then beneficial in the treatment of osteoporosis.

A compound having an activating effect on CaSR (CaSR agonist), that is, a compound which selectively acts on CaSR to mimic or strengthen the action of Ca²⁺, is called a calcimimetic. On the other hand, a compound having an antagonistic effect on CaSR (CaSR antagonist, that is, a compound which suppresses or inhibits the action of Ca²⁺), is called a calcilytic.

The calcium-sensing receptor has recently been found to be a potent target for developing therapeutic options such as using calcimimetics for treatment of diarrhea. [Osigweh et al, J American Coll. of Surgeons, V201, Issue 3, suppl 1, September 2005, p17.]

Calcimimetics have been shown to be commercially useful for the treatment of hyperparathyroidism (HPT): The calcimimetic compound Cinacalcet® [Balfour, J. A. B. et al. Drugs (2005) 65(2), 271-281; Linberg et. al. J. Am. Soc. Nephrol (2005), 16, 800-807, Clinical Therapeutics (2005), 27(11), 1725-1751] is commercially available for the treatment of secondary HPT in chronic kidney disease patients on dialysis and for the treatment of primary HPT in patients with parathyroid carcinoma. Thus, proof of concept for activators of calcium sensing receptor (CaSR) in humans has been achieved and the clinical relevance is well established.

Other calcimimetic compounds were for example described in WO02/059102, WO98/001417, WO05/065050, WO 05/34928, WO03/099814, WO03/099776, WO00/21910, WO01/34562, WO01/090069, WO97/41090, U.S. Pat. No. 6,001,884, WO96/12697, EP1203761, WO95/11221, WO93/04373, EP1281702, WO02/12181, WO04/56365, WO04/069793, WO04/094362, US2004242602, WO04/106280, WO04/106295, WO04/106296, WO05/068433, WO05/115975, EP 1757582, WO 2009/051718 and WO2010/021351.

### SUMMARY OF THE INVENTION

It has surprisingly been found that the novel compounds of the present invention are modulators, e.g. activators or agonists of the human calcium sensing receptor (CaSR) and may thus be useful in the treatment or prophylaxis of a number of diseases or physiological disorders involving modulation of CaSR activity.

Accordingly, the present invention relates to a compound of general formula I

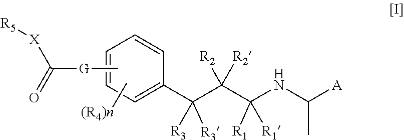

wherein
A is C₁₋₁₀heteroaryl, C₆₋₁₄aryl or C₆₋₁₀heterocycloalkylaryl, optionally substituted by one or more substituents independently at each occurrence selected from the group consisting of halogen, hydroxy, trifluoromethyl, amino, C₁₋₆-alkyl, C₂₋₆-alkenyl, C₁₋₆alkyloxy, or C₁₋₆-alkenyloxy, wherein said C₁₋₆-alkyl, C₂₋₆-alkenyl, C₁₋₆alkyloxy, or C₂₋₆-alkenyloxy may be optionally substituted by one or more substituents selected from halogen, hydroxy, trifluoromethyl and NH₂;

R₁, R₁′, R₂, R₂′, R₃ and R₃′, independently of each other, are selected from the group consisting of hydrogen, halogen, hydroxy, trifluoromethyl, amino, C₁₋₆-alkyl, C₂₋₆-alkenyl, C₁₋₆-alkyloxy, and C₂₋₆-alkenyloxy;

R₄, independently at each occurrence, is selected from the group consisting of hydrogen, halogen, hydroxy, trifluoromethyl, amino, C₁₋₆-alkyl, C₂₋₆-alkenyl, C₁₋₆-alkyloxy, and C₁₋₆-alkenyloxy, wherein said C₁₋₆-alkyl, C₂₋₆-alkenyl, C₁₋₆-alkyloxy, or C₂₋₆-alkenyloxy may be optionally substituted by one or more substituents selected from halogen, hydroxy, trifluoromethyl and NH₂;

n is an integer from 0 to 3;

R₅ is hydrogen or is selected from the group consisting of C₁₋₆-alkyl, C₂₋₆-alkenyl, C₁₋₆-alkyloxy, and C₁₋₆-alkenyloxy, C₁₋₅-heterocyclyl, C₁₋₅-heterocyclyl-C₁₋₆-alkyl and C₁₋₅-heterocyclyl-C₂₋₆-alkenyl, optionally substituted by one or more substituents independently at each occurrence selected from the group consisting of halogen, hydroxy, trifluoromethyl, amino, C₁₋₆-alkyl, C₂₋₆-alkenyl, C₁₋₆-alkyloxy, and C₁₋₆-alkenyloxy, wherein said C₁₋₆-alkyl, C₂₋₆-alkenyl, C₁₋₆-alkyloxy, or C₂₋₆-alkenyloxy may be optionally substituted by one or more substituents selected from halogen, hydroxy, trifluoromethyl and NH₂, or

R₅ is C₃₋₄-alkylene and together with —C(O)—O forms a 5 or 6 membered cyclic or heterocyclic ring attached to G;

X is O;

G is a direct bond or is selected from the group consisting of C₁₋₆-alkylene, —O—C₁₋₆alkylene, C₂₋₆-alkenylene, —O—C₂₋₆-alkenylene, C₁₋₅-heterocyclylene, —R₆—CONH—R₈, or —R₂—CO—R₉;

R₆, R₇, R₈ and R₉, independently of each other, is a direct bond or is C₁₋₆-alkylene, C₂₋₆-alkenylene, C₃₋₆-cycloalkylene or C₁₋₅-heterocyclylene, wherein said C₁₋₆-alkylene, C₂₋₆-alkenylene, C₃₋₆-cycloalkylene or C₁₋₅-heterocyclylene is optionally substituted by one or more substituents independently at each occurrence selected from the group consisting of halogen, hydroxy, trifluoromethyl, amino, C₁₋₆-alkyl, C₂₋₆-alkenyl, C₁₋₆-alkyloxy, or C₁₋₆-alkenyloxy, wherein said C₁₋₆-alkyl, C₂₋₆-alkenyl, C₁₋₆-alkyloxy, or C₂₋₆-alkenyloxy may be optionally substituted by one or more substituents selected from halogen, hydroxy, trifluoromethyl and NH₂;

as well as stereoisomers, pharmaceutically acceptable salts, solvates, hydrates, or in vivo hydrolysable esters thereof.

In another aspect the invention relates to a compound of general formula Ia

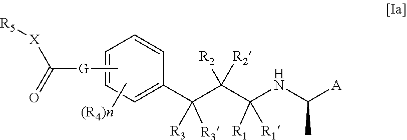

wherein

A is C₁₋₁₀heteroaryl, C₆₋₁₄aryl or C₆₋₁₀heterocycloalkylaryl, optionally substituted by one or more substituents independently at each occurrence selected from the group consisting of halogen, hydroxy, trifluoromethyl, amino, C₁₋₆-alkyl, C₂₋₆-alkenyl, C₁₋₆-alkyloxy, or C₁₋₆-alkenyloxy, wherein said C₁₋₆-alkyl, C₂₋₆-alkenyl, C₁₋₆alkyloxy, or C₂₋₆-alkenyloxy may be optionally substituted by one or more substituents selected from halogen, hydroxy, trifluoromethyl and NH₂;

R₁, R₁′, R₂, R₂′, R₃ and R₃′, independently of each other, are selected from the group consisting of hydrogen, halogen, hydroxy, trifluoromethyl, amino, C₁₋₆-alkyl, C₂₋₆-alkenyl, C₁₋₆-alkyloxy, and C₂₋₆-alkenyloxy;

R₄, independently at each occurrence, is selected from the group consisting of hydrogen, halogen, hydroxy, trifluoromethyl, amino, C₁₋₆-alkyl, C₂₋₆-alkenyl, C₁₋₆-alkyloxy, and C₁₋₆-alkenyloxy, wherein said C₁₋₆-alkyl, C₂₋₆-alkenyl, C₁₋₆-alkyloxy, or C₂₋₆-alkenyloxy may be optionally substituted by one or more substituents selected from halogen, hydroxy, trifluoromethyl and NH₂;

n is an integer from 0 to 3;

R₅ is hydrogen or is selected from the group consisting of C₁₋₆-alkyl, C₂₋₆-alkenyl, C₁₋₆-alkyloxy, and C₁₋₆-alkenyloxy, C₁₋₅-heterocyclyl, C₁₋₅-heterocyclyl-C₁₋₆-alkyl and C₁₋₅-heterocyclyl-C₂₋₆-alkenyl, optionally substituted by one or more substituents independently at each occurrence selected from the group consisting of halogen, hydroxy, trifluoromethyl, amino, C₁₋₆-alkyl, C₂₋₆-alkenyl, C₁₋₆alkyloxy, and C₁₋₆-alkenyloxy, wherein said C₁₋₆-alkyl, C₂₋₆-alkenyl, C₁₋₆-alkyloxy, or C₂₋₆-alkenyloxy may be optionally substituted by one or more substituents selected from halogen, hydroxy, trifluoromethyl and NH₂, or

R₅ is C₃₋₄-alkylene and together with the adjacent —C(O)—O forms a 5 or 6 membered cyclic or heterocyclic ring attached to G;

X is O;

G is a direct bond or is selected from the group consisting of C₁₋₆-alkylene, —O—C₁₋₆-alkylene, C₂₋₆-alkenylene, —O—C₂₋₆-alkenylene, C₁₋₅-heterocyclylene, —R₆—CONH—R₈, or —R₇—CO—R₉;

R₆, R₇, R₈ and R₉, independently of each other, is a direct bond or is C₁₋₆-alkylene, C₂₋₆-alkenylene, C₃₋₆-cycloalkylene or C₁₋₅-heterocyclylene, wherein said C₁₋₆-alkylene, C₂₋₆-alkenylene, C₃₋₆-cycloalkylene or C₁₋₅-heterocyclylene is optionally substituted by one or more substituents independently at each occurrence selected from the group consisting of halogen, hydroxy, trifluoromethyl, amino, C₂₋₆-alkenyl, C₁₋₆-alkyloxy, or C₁₋₆-alkenyloxy, wherein said C₁₋₆-alkyl, C₂₋₆-alkenyl, C₁₋₆-alkyloxy, or C₂₋₆-alkenyloxy may be optionally substituted by one or more substituents selected from halogen, hydroxy, trifluoromethyl and NH₂;

as well as stereoisomers, pharmaceutically acceptable salts, solvates, hydrates, or in vivo hydrolysable esters thereof.

The compounds of the present invention may for example be useful in the treatment of complications associated with chronic kidney disease, such as hyperparathyroidism, e.g. primary and/or secondary hyperparathyroidism, or tertiary hyperparathyroidism. Other complications associated with chronic kidney disease are anemia, cardiovascular diseases, and the compounds of the present invention are also believed to have a beneficial effect on these diseases. The compounds of the present invention may furthermore be useful for promoting osteogenesis and treating or preventing osteoporosis, such as steroid induced, senile and post menopausal osteoporosis; osteomalacia and related bone disorders, or for the prevention of bone loss post renal transplantation, or in rescue therapy pre-parathyroidectomy.

It is presently believed that the compounds of the present invention may have advantageous pharmacokinetic or pharmacodynamic properties, such as oral bioavailability, in comparison to known structurally related compounds.

The compounds of formula I and Ia according to the present invention all contain a carboxylic acid, or a derivative thereof which will set free the carboxylic acid upon absorption in the body.

This feature imparts on the molecules their low binding affinity (IC-50 above 1 μM) to the hERG (human Ether á-go-go Related Gene) proteins and also to the hemoproteins Cytochrome P450 (CYP)'s, eg CYP3A4 and CYP2D6 and are thus considered more safe and with smaller risk of drug-drug interactions. This can be attributed to the decrease in lipophilicity caused by the introduction of the carboxylic acid.

In another aspect, the invention relates to the compound of general formula I or Ia as defined above for use as a medicament in therapy.

In another aspect, the invention relates to the compound of general formula I or Ia as defined above for use in the prophylaxis, treatment or amelioration of physiological disorders or diseases associated with disturbances of CaSR activity, such as hyperparathyroidism.

In another aspect, the invention relates to the use of a compound of general formula I or Ia as defined above for the manufacture of a medicament for the prophylaxis, treatment or amelioration of physiological disorders or diseases associated with disturbances of CaSR activity, such as hyperparathyroidism.

In yet another aspect, the invention relates to a pharmaceutical composition comprising a compound of formula I or Ia or a pharmaceutically acceptable salt, solvate, or in vivo hydrolysable ester thereof together with a pharmaceutically acceptable excipient or vehicle.

In a further aspect, the invention relates to a method of preventing, treating or ameliorating parathyroid carcinoma, parathyroid adenoma, primary parathyroid hyperplasia, cardiac, renal or intestinal disfunctions, diseases of the central nervous system, chronic renal failure, chronic kidney disease, polycystic kidney disorder, podocyte-related diseases, primary hyperparathyroidism, secondary hyperparathyroidism, tertiary hyperparathyroidism, anemia, cardiovascular diseases, renal osteodystrophy, osteitis fibrosa, adynamic bone disease, osteoporosis, steroid induced osteoporosis, senile osteoporosis, post menopausal osteoporosis, osteomalacia and related bone disorders, bone loss post renal transplantation, cardiovascular diseases, gastrointestinal diseases, endocrine and neurodegenerative diseases, cancer, Alzheimer's disease, IBS, IBD, malassimilation, malnutrition, abnormal intestinal motility such as diarrhea, vascular calcification, abnormal calcium homeostasis, hypercalcemia, or renal bone diseases, the method comprising administering to a patient in need thereof an effective amount of a compound of general formula I or Ia as defined above, optionally in combination or as supplement with an active vitamin-D sterol or vitamin-D derivative, such as 1-α-hydroxycholecalciferol, ergocalciferol, cholecalciferol, 25-hydroxycholecalciferol, 1-α-25-dihydroxycholecalciferol, or in combination or as supplement with phosphate binders, estrogens, calcitonin or biphosphonates.

### DETAILED DESCRIPTION OF THE INVENTION

### Definitions

The term “heteroaryl” is intended to include radicals of heterocyclic aromatic rings, comprising 1-4 heteroatoms (selected from O, S and N) and 1-10 carbon atoms, such as 1-3 heteroatoms and 1-6 carbon atoms, such as 1-2 heteroatoms and 1-5 carbon atoms, such as 1-2 heteroatoms and 2-4 carbon atoms, in particular 5- or 6-membered rings with 1-4 heteroatoms or 1-2 heteroatoms selected from O, S and N, or such as 1-4 heteroatoms and 6-10 carbon atoms, in particular 9-membered rings with 1-2 heteroatoms, e.g. pyridyl, tetrazolyl, thiazolyl, imidazolyl, pyrazolyl, oxazolyl, oxadiazolyl, thiophenyl, 1,2,4-triazolyl, isoxazolyl, thienyl, pyrazinyl, pyrimidinyl, [1,2,3]triazolyl, quinolyl, indazolyl, isothiazolyl, benzo[b]thiophene or indolyl.

The term “cycloalkyl” is intended to indicate a saturated cycloalkane radical or ring, comprising 2-12 carbon atoms, such as 3-6 carbon atoms, such as 4-5 carbon atoms, e.g. cyclopropyl, cyclobutyl, cyclopentyl, and cyclohexyl.

The term “cycloalkylene” is intended to indicate a divalent cycloalkyl group as defined herein.

The term “heterocycloalkyl” is intended to indicate a cycloalkyl radical as defined above, in particular 5- or 6-membered rings, including polycyclic radicals, comprising 1-4 heteroatoms, preferably 1-3 heteroatoms, selected from O, N, or S, e.g. tetrahydropyranyl, morpholino, morpholinyl, imidazolidinyl, dioxolanyl, piperidyl, piperazinyl, pyrrolidinyl, piperidino, piperidinyl, azetidino, azetidinyl and tetrahydrofuryl.

The term “heterocycloalkylaryl” is intended to include radicals of heterocycloalkyl rings, in particular 5- or 6-membered rings, comprising 1-5 carbon atoms and 1-4 hetero atoms (selected form O, S and N), such as 1-4 carbon atoms and 1-3 hetero atoms, preferably 2-3 carbon atoms and 1-2 hetero atoms selected from O, S, or N, the heterocycloalkyl ring being fused with one or more aromatic carbocyclic rings comprising 6-20 carbon atoms, such as 6-14 carbon atoms, preferably 6-10 carbon atoms, in particular 5-, 6- or 10 membered rings, such as phenyl or naphthyl, e.g. benzodioxol.

The term “aryl” is intended to indicate a radical of aromatic carbocyclic rings comprising 6-20 carbon atoms, such as 6-14 carbon atoms, preferably 6-10 carbon atoms, in particular 5- or 6-membered rings, optionally fused carbocyclic rings with at least one aromatic ring, such as phenyl, naphthyl, e.g. 1-naphthyl, indenyl, indanyl and tetrahydro-naphthalene.

The term “heterocyclyl” is intended to include “heteroaryl”, “heterocycloalkyl” and “heterocycloalkylaryl”.

The term “heterocyclylene” is intended to indicate a divalent heterocyclyl group as defined herein.

The term “halogen” is intended to indicate a substituent from the r^{h} main group of the periodic table, preferably fluoro, chloro and bromo.

In the present context, the term “alkyl” is intended to indicate the radical obtained when one hydrogen atom is removed from a hydrocarbon. Said alkyl comprises 1-6, preferably 1-4, such as 2-3, carbon atoms. The term includes the subclasses normal alkyl (n-alkyl), secondary and tertiary alkyl, such as methyl, ethyl, n-propyl, isopropyl, n-butyl, isobutyl, sec.-butyl, tert.-butyl, pentyl, isopentyl, hexyl and isohexyl.

The term “alkylene” is intended to indicate a divalent saturated aliphatic hydrocarbyl group preferably having from 1 to 6 and more preferably 1 to 3 carbon atoms that are either straight-chained or branched. This term is exemplified by groups such as methylene (—CH₂—), ethylene (—CH₂CH₂—), n-propylene (—CH₂CH₂CH₂—), iso-propylene (—C((CH₃)₂)—), —CH₂CH(CH₃)—, —CH(CH₃)CH₂—, or —C(CH₃)₂CH₂— and the like.

The term “alkenyl” is intended to indicate a mono-, di-, or tri-unsaturated hydrocarbon radical comprising 2-6 carbon atoms, in particular 2-4 carbon atoms, such as 2-3 carbon atoms, e.g. ethenyl, allyl, propenyl, butenyl, pentenyl, or hexenyl.

The term “alkenylene” is intended to indicate a divalent mono-unsaturated aliphatic hydrocarbyl group preferably having from 2 to 6 and more preferably 2 to 4 carbon atoms that are either straight-chained or branched, e.g. ethenylene (—CH═CH—).

The term “heterocyclyl-alkyl” is intended to include a heterocyclyl radical as defined herein connected to an alkyl radical as defined herein, e.g. morpholino-ethyl.

The term “heterocyclyl-alkenyl” is intended to include a heterocyclyl radical as defined herein connected to an alkenyl radical as defined herein, e.g. piperidino-ethenyl.

The term “hydroxyalkyl” is intended to indicate an alkyl radical as defined above, wherein one, two, three or more hydrogen atoms are replaced by hydroxyl, e.g. hydroxypropyl.

The term “haloalkyl” is intended to indicate an alkyl radical as defined above, wherein one, two, three or more hydrogen atoms are replaced by halogen, same or different, such as bromo, iodo, chloro and/or fluoro, e.g. chloromethyl.

The term “alkyloxy” is intended to indicate a radical of the formula —OR, wherein R is alkyl as indicated above, e.g. methoxy, ethoxy, n-propoxy, isopropoxy, butoxy, etc.

The term “alkenyloxy” is intended to indicate a radical of the formula —OR, wherein R is alkenyl as indicated above, e.g. ethenyloxy etc.

The term “amino” is intended to indicate a radical of the formula —NRR′, wherein R and R′ independently represent hydrogen, alkyl, alkenyl, or cycloalkyl, as indicated above, e.g. —NH₂, dimethylamino, methylamino, diethylamino, cyclohexylamino, tert-butylamino, ethylmethylamino, ethylamino or ethenylamino.

The term “cycloalkenyl” is intended to indicate mono-, or di-unsaturated non-aromatic cyclic hydrocarbon radicals, comprising 2-10 carbon atoms, such as 3-6 carbon atoms, such as 4-5 carbon atoms, e.g. cyclopropenyl, cyclobutenyl, cyclopentenyl, or cyclohexenyl.

The term “heterocycloalkenyl” is intended to indicate a cycloalkenyl radical as defined above, including polycyclic radicals, comprising 1-4 heteroatoms, preferably 1-3 heteroatoms, selected from O, N, or S, e.g. 1,6-dihydropyridinyl, 4,5-dihydro-1H-[1,2,4]-triazolyl, 4,5-dihydro-oxazolyl, 1-H-pyrazolyl, or 4,5-dihydro-isoxazolyl.

The term “pharmaceutically acceptable salt” is intended to indicate salts prepared by reacting a compound of formula I or Ia with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxy ethanesulfonic acid, toluenesulfonic, sulfamic or fumaric acid. Pharmaceutically acceptable salts of compounds of formula I or Ia may also be prepared by reaction with a suitable base such as sodium hydroxide, potassium hydroxide, magnesium hydroxide, calcium hydroxide, ammonia, or suitable non-toxic amines, such as lower alkylamines, for example triethylamine, hydroxy-lower alkylamines, for example 2-hydroxyethylamine, bis-(2-hydroxyethyl)-amine, cycloalkylamines, for example dicyclohexylamine, or benzylamines, for example N,N′-dibenzylethylenediamine, and dibenzylamine, or L-arginine or L-lysine.

The term “solvate” is intended to indicate a species formed by interaction between a compound, e.g. a compound of formula I or Ia, and a solvent, e.g. alcohol, glycerol or water, wherein said species are in a solid form. When water is the solvent, said species is referred to as a hydrate.

The term “pharmaceutically acceptable ester” is intended to indicate easily hydrolysable esters, i.e. in vivo hydrolysable esters of the compounds of formula I or Ia such as alkanoyloxyalkyl, aralkanoyloxyalkyl, aroyloxyalkyl, e.g. acetoxymethyl, pivaloyloxymethyl, benzoyloxymethyl esters and the corresponding 1′-oxyethyl derivatives, or alkoxycarbonyloxyalkyl esters, e.g. methoxycarbonyloxymethyl esters and ethoxycarbonyloxymethyl esters and the corresponding 1′-oxyethyl derivatives, or lactonyl esters, e.g. phthalidyl esters, or dialkylaminoalkyl esters, e.g. dimethylaminoethyl esters Said esters can be converted into the corresponding free carboxylic acids by chemical means such as hydrolysis or by enzymological means. Such esters may be prepared by conventional methods known to persons skilled in the art, such as the method disclosed in GB patent No. 1 490 852 incorporated herein by reference.

Compounds of formula I or Ia may comprise asymmetrically substituted (chiral) carbon atoms and carbon-carbon double bonds which may give rise to the existence of isomeric forms, e.g. enantiomers, diastereomers and geometric isomers. The present invention includes all such isomers, either in pure form or as mixtures thereof. Pure stereoisomeric forms of the compounds and the intermediates of this invention may be obtained by the application of procedures known in the art. Diastereomers may be separated by physical separation methods such as selective crystallization and chromatographic techniques, e.g. liquid chromatography using chiral stationary phases. Enantiomers may be separated from each other by the selective crystallization of their diastereomeric salts with optically active acids. Alternatively, enantiomers may be separated by chromatographic techniques using chiral stationary phases. Said pure stereoisomeric forms may also be derived from the corresponding pure stereoisomeric forms of the appropriate starting materials, provided that the reaction occurs stereoselectively or stereospecifically. Preferably, if a specific stereoisomer is desired, said compound will be synthesized by stereoselective or stereospecific methods of preparation. These methods will advantageously employ chirally pure starting materials. Likewise, pure geometric isomers may be obtained from the corresponding pure geometric isomers of the appropriate starting materials. A mixture of geometric isomers will typically exhibit different physical properties, and they may thus be separated by standard chromatographic techniques well-known in the art.

The present invention further includes prodrugs of compounds of general formula I or Ia, i.e. derivatives such esters, ethers, complexes or other derivatives which undergo a biotransformation in vivo before exhibiting their pharmacological effects.

The compounds of formula I or Ia may be obtained in crystalline form either directly by concentration from an organic solvent or by crystallisation or re-crystallisation from an organic solvent or mixture of said solvent and a co-solvent that may be organic or inorganic, such as water. The crystals may be isolated in essentially solvent-free form or as a solvate, such as a hydrate. The invention covers all crystalline modifications and forms and also mixtures thereof.

### Embodiments

In an embodiment of the compound of the formula I or Ia according to the present invention A is naphthyl.

In an embodiment of the compound of the formula I or Ia according to the present invention A is 4-fluoro-3-methoxy-phenyl.

In an embodiment of the compound of the formula I or Ia according to the present invention R₁, R₁′, R₂, R₂′, R₃ and R₃′ are all hydrogen.

In an embodiment of the compound of the formula I or Ia according to the present invention at least one of R₁, R₁′, R₂, R₂′, R₃ and R₃′ is methyl.

In an embodiment of the compound of the formula I or Ia according to the present invention at least one R₁, R₁′, R₂, R₂′, R₃ and R₃′ is hydroxy.

In an embodiment of the compound of the formula I or Ia according to the present invention n is 0.

In an embodiment of the compound of the formula I or Ia according to the present invention n is 1.

In an embodiment of the compound of the formula I or Ia according to the present invention R₄ is hydroxy.

In an embodiment of the compound of the formula I or Ia according to the present invention R₄ is F.

In an embodiment of the compound of the formula I or Ia according to the present invention R₄ is trifluoromethyl.

In an embodiment of the compound of the formula I or Ia according to the present invention R₅ is hydrogen.

In an embodiment of the compound of the formula I or Ia according to the present invention R₅ is selected from the group consisting of methyl, ethyl, n-propyl, isopropyl, n-butyl, isobutyl, sec-butyl and tert-butyl.

In an embodiment of the compound of the formula I or Ia according to the present invention R₅ is morpholino-ethyl.

In an embodiment of the compound of the formula I or Ia according to the present invention R₅ together with the adjacent —C(O)—O— represents 2-oxo-tetrahydrofuranyl.

In an embodiment of the compound of the formula I or Ia according to the present invention G is a direct bond.

In an embodiment of the compound of the formula I or Ia according to the present invention G is ethylene or methoxy.

In an embodiment of the compound of the formula I or Ia according to the present invention G is isopropylene, O-isopropylene, or tetrahydropyranylene.

In an embodiment of the compound of the formula I or Ia according to the present invention G is —R₆—CONH—R₈.

In an embodiment of the compound of the formula I or Ia according to the present invention R₆ is a direct bond.

In an embodiment of the compound of the formula I or Ia according to the present invention R₆ is C₁₋₅-alkylene or heterocyclylene.

In an embodiment of the compound of the formula I or Ia according to the present invention R₆ is isopropylene or tetrahydropyranylene.

In an embodiment of the compound of the formula I or Ia according to the present invention R₈ is C₁₋₆-alkylene.

In an embodiment of the compound of the formula I or Ia according to the present invention R₈ is methylene, ethylene, n-propylene or isopropylene, optionally substituted with one or more substituents selected from halogen, hydroxy, amino, trifluoromethyl, C₁-C₄-alkyl, C₁₋₄-alkoxy or hydroxy-C₁-C₄-alkyl.

In an embodiment of the compound of the formula I or Ia according to the present invention G is —R₇—CO—R₉.

In an embodiment of the compound of the formula I or Ia according to the present invention R₇ is C₁₋₆-alkylene or heterocyclylene.

In an embodiment of the compound of the formula I or Ia according to the present invention R₇ is isopropylene or tetrahydropyranylene.

In an embodiment of the compound of the formula I or Ia according to the present invention R₉ is heterocyclylene.

In an embodiment of the compound of the formula I or Ia according to the present invention R₉ is piperidino or azetidino.

In an embodiment of the compound of the formula I or Ia according to the present invention, said compound is selected from:
Ethyl(R)-3-(3-{1-naphthalen-1-yl-ethylamino}-propyl)-benzoate, hydrochloride (compound 1001),
(R)-2-Hydroxy-5-[3-(1-naphthalen-1-yl-ethylamino)-propyl]-benzoic acid methyl ester hydrochloride (compound 1002),
(R)-2-Hydroxy-5-[3-(1-naphthalen-1-yl-ethylamino)-propyl]-benzoic acid (compound 1003),
Methyl(R)-2-[3-(1-naphthalen-1-yl-ethylamino)-propyl]-benzoate hydrochloride (compound 1004),
2-[3-((R)-1-Naphthalen-1-yl-ethylamino)-propyl]benzoic acid hydrochloride (compound 1005),
(R)-2-Fluoro-5-[3-(1-naphthalen-1-yl-ethylamino)-propyl]-benzoic acid (compound 1006),
4-[3-((R)-1-Naphthalen-1-yl-ethylamino)-butyl]-benzoic acid methyl ester (compound 1007),
tert-Butyl(R)-(2,2-Dimethyl-2-{4-[3-(1-naphthalen-1-yl-ethylamino)-propyl]-phenoxy}-acetoylamino)-acetate (compound 1008),
(R)-(2,2-Dimethyl-2-{4-[3-(1-naphthalen-1-yl-ethylamino)-propyl]-phenoxy}-acetoylamino)-acetic acid, hydrochloride (compound 1009),
(R)-4-[3-(1-Naphthalen-1-yl-ethylamino)-propyl]-benzoic acid (compound 1010),
(R)-3-(3-{1-naphthalen-1-yl-ethylamino}-propyl)-benzoic acid (compound 1011),
(R)-4-[3-(1-Naphthalen-1-yl-ethylamino)-propyl]-benzoic acid 2-morpholin-4-yl-ethyl ester hydrochloride (compound 1012),
Ethyl 4-(2-hydroxy-3-{(R)-1-naphthalen-1-yl-ethylamino}-propyl)-benzoate (compound 1013),
4-(2-hydroxy-3-{(R)-1-naphthalen-1-yl-ethylamino}-propyl)-benzoic acid (compound 1014),
(R)-2′,2′-Dimethyl-2′-{4-[3-(1-naphthalen-1-yl-ethylamino)-propyl]-phenyl}-acetic acid, hydrochloride (compound 1015),
(R)-4-(4-[3-(1-naphthalen-1-yl-ethylamino)-propyl]-phenyl)-tetrahydropyrane-4-carboxylic acid (compound 1016),
4-[3-((R)-1-Naphthalen-1-yl-ethylamino)-propyl]-N—((R)-2-oxo-tetrahydro-furan-3-yl)-benzamide (compound 1017),
4-Hydroxy-2R-{4-[3-((R)-1-naphthalen-1-yl-ethylamino)-propyl]-benzoylamino}-butyric acid (compound 1018),
(R)-Methyl (2,2-dimethyl-2-{4-[3-(1-naphthalen-1-yl-ethylamino)-propyl]-phenyl}-acetoylamino)-acetate hydrochloride (compound 1019),
(R)-(2,2-Dimethyl-2-{4-[3-(1-naphthalen-1-yl-ethylamino)-propyl]-phenyl}-acetoylamino)-acetic acid (compound 1020),
(R)-3-(2,2-Dimethyl-2-{4-[3-(1-naphthalen-1-yl-ethylamino)-propyl]-phenyl}-acetoylamino)-propionic acid (compound 1021),
(R)-1-(2,2-Dimethyl-2-{4-[3-(1-naphthalen-1-yl-ethylamino)-propyl]-phenyl}-acetoyl)-piperidine-4-carboxylic acid; hydrochloride (compound 1022),
(R)-2-Methyl-2-{4-[3-(1-naphthalen-1-yl-ethylamino)-propyl]-benzoylamino}-propionic acid methyl ester (compound 1023),
(R)-2-Methyl-2-{4-[3-(1-naphthalen-1-yl-ethylamino)-propyl]-benzoylamino}-propionic acid (compound 1024),
(R)-[(4-{4-[3-(1-Naphthalen-1-yl-ethylamino)-propyl]-phenyl}-tetrahydro-pyran-4-carbonyl)-amino]-acetic acid methyl ester (compound 1025),
(R)-[(4-{4-[3-(1-Naphthalen-1-yl-ethylamino)-propyl]-phenyl}-tetrahydro-pyran-4-carbonyl)-amino]-acetic acid (compound 1026),
(R)-3-[(4-{4-[3-(1-Naphthalen-1-yl-ethylamino)-propyl]-phenyl}-tetrahydro-pyran-4-carbonyl)-amino]-propionic acid methyl ester (compound 1027),
(R)-3-[(4-{4-[3-(1-Naphthalen-1-yl-ethylamino)-propyl]-phenyl}-tetrahydro-pyran-4-carbonyl)-amino]-propionic acid (compound 1028),
(R)-1-(4-{4-[3-(1-Naphthalen-1-yl-ethylamino)-propyl]-phenyl}-tetrahydro-pyran-4-carbonyl)-azetidine-3-carboxylic acid methyl ester (compound 1029),
(R)-1-(4-{4-[3-(1-Naphthalen-1-yl-ethylamino)-propyl]-phenyl}-tetrahydro-pyran-4-carbonyl)-azetidine-3-carboxylic acid (compound 1030),
(R)-4-[3-[[1-(4-fluoro-3-methoxy-phenyl)ethyl]amino]-propyl]benzoic acid (compound 1031),
(R)-3-{3-[1-(4-Fluoro-3-methoxy-phenyl)-ethylamino]-propyl}-benzoic acid (compound 1032),
(R)-{4-[3-((1-Naphthalen-1-yl-ethylamino)-propyl]-phenoxy}-acetic acid (compound 1033),
(R)-3-{4-[3-(1-Naphthalen-1-yl-ethylamino)-propyl]-phenyl}-propionic acid (compound 1034)
(R)-3-(3-{3-[1-(4-Fluoro-3-methoxy-phenyl)-ethylamino]-propyl}-phenyl)-propionic acid (compound 1035),
(R)-3-{3-[3-(1-Naphthalen-1-yl-ethylamino)-propyl]-phenyl}-propionic acid (compound 1036),
(R)-2-[2-fluoro-4-[3-[[1-(4-fluoro-3-methoxy-phenyl)ethyl]amino]-propyl]phenoxy]acetic acid (compound 1037),
(R)-{2-Fluoro-4-[3-(1-naphthalen-1-yl-ethylamino)-propyl]-phenoxy}-acetic acid (compound 1038),
(R)-3-{2-Fluoro-4-[3-(1-naphthalen-1-yl-ethylamino)-propyl]-phenyl}-propionic acid (compound 1039),
(R)-3-(2-Fluoro-4-{3-[1-(4-fluoro-3-methoxy-phenyl)-ethylamino]-propyl}-phenyl)-propionic acid (compound 1040), and
(R)-3-[4-[3-[[1-(1-naphthyl)ethyl]amino]propyl]-2-(trifluoromethyl)-phenyl]propanoic acid (compound 1041).

Specific examples of intermediates for the preparation of compounds of formula I may be selected from the group consisting of:
(R)-(1-Naphthalen-1-yl-ethyl)-prop-2-ynyl-amine (preparation 1),
Ethyl(R)-3-[3-(1-naphthalen-1-yl-ethylamino)-prop-1-ynyl]-benzoate hydrochloride (preparation 2),
Methyl(R)-2-hydroxy-5-[3-(1-naphthalen-1-yl-ethylamino)-prop-1-ynyl]-benzoate hydrochloride (preparation 3),
tert-Butyl 2-(4-iodophenoxy)-2,2-dimethylacetate (preparation 4),
tert-Butyl(R)-(2,2-Dimethyl-2-{4-[3-(1-naphthalen-1-yl-ethylamino)-1-propyn-1-yl]-phenoxy}-acetoylamino)-acetate (preparation 5),
(R)-4-[3-(1-Naphthalen-1-yl-ethylamino)-propyn-1-yl]-benzoic acid (preparation 6),
Ethyl (±)-4-(oxiranylmethyl)-benzoate (preparation 7),
Methyl(R)-2′,2′-Dimethyl-2′-{4-[3-(1-naphthalen-1-yl-ethylamino)-propyn-1-yl]-phenyl}-acetate (preparation 8),
(R)-2′,2′-Dimethyl-2′-{4-[3-(1-naphthalen-1-yl-ethylamino)-propyn-1-yl]-phenyl}-acetic acid, hydrochloride (preparation 9),
4-(4-Iodophenyl)-tetrahydropyrane-4-carbonitrile (preparation 10),
(R)-4-(4-[3-(1-naphthalen-1-yl-ethylamino)-prop-1-ynyl]-phenyl)-tetrahydropyrane-4-carbonitrile (preparation 11),
(R)-4-(4-[3-(1-naphthalen-1-yl-ethylamino)-propyl]-phenyl)-tetrahydropyrane-4-carbonitrile (preparation 12),
(R) —N-[1-(4-fluoro-3-methoxy-phenyl)ethyl]-prop-2-yn-1-amine (Preparation 13),
4-{3-[(1R)-1-(4-Fluoro-3-methoxy-phenyl)-ethylamino]-prop-1-ynyl}-benzoic acid (preparation 14),
(4-Iodo-phenoxy)-acetic acid methyl ester (preparation 15),
3-(4-Iodo-phenyl)-acrylic acid methyl ester (preparation 16),
3-(3-Iodo-phenyl)-acrylic acid methyl ester (preparation 17),
2-Fluoro-4-iodophenol (preparation 18),
(2-Fluoro-4-iodo-phenoxy)-acetic acid ethyl ester (preparation 19),
Methyl 2-Fluoro-4-bromophenylpropanoate (preparation 20),
Methyl 2-fluoro-4-iodophenylpropanoate (preparation 21),
{4-[3-((1R)-1-Naphthalen-1-yl-ethylamino)-prop-1-ynyl]-phenoxy}-acetic acid methyl ester (preparation 22),
3-(4-Bromo-2-trifluoromethyl-phenyl)-acrylic acid methyl ester (preparation 23),
   or
3-(4-Iodo-2-trifluoromethyl-phenyl)-acrylic acid methyl ester (preparation 24).

### Methods of Preparation

The compounds of general formula I or Ia may be prepared in a number of ways well known to those skilled in the art of organic synthesis. The compounds of formula I or Ia may be synthesised using the methods outlined below, together with methods known in the art of synthetic organic chemistry, or variations thereof as appreciated by those skilled in the art. Preferred methods include, but are not limited to, those described below.

The compounds of formula I or Ia may be prepared by techniques and procedures readily available to one of ordinary skill in the art, for example by following the procedures as set forth in the following schemes. The reactions are performed in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognised by one skilled in the art. It is understood by one skilled in the art of organic synthesis that the functionalities present on various portions of the starting molecules in a reaction must be compatible with the reagents and reactions proposed. Not all compounds of formula I or Ia falling into a given class may be compatible with some of the reaction conditions required in some of the methods described. Such restrictions to the substituents which are compatible with the reaction conditions will be readily apparent to one skilled in the art and alternative methods can be used.

The schemes described in this section are not intended to limit the scope of the invention in any way. All substituents, unless otherwise indicated, are previously defined. The reagents and starting materials are either available from commercial suppliers or prepared by methods known to one of ordinary skilled in the art following procedures set forth in references such as Fieser and Fieser's Reagents for Organic Synthesis, Volumes 1-22 (John Wiley and Sons, 2004); Rodd's Chemistry of Carbon Compounds, Volumes 1-5 and Supplements (Elsevier Science Publishers, 2000); Organic Reactions, Volumes 1-64 (John Wiley and Sons, 2004); March's Advanced Organic Chemistry (John Wiley and Sons, 5^{th} Edition) and Larock's Comprehensive Organic Transformations (VCH Publishers Inc., 1999). These schemes are merely illustrative of some methods by which the compounds of this invention can be synthesised, and various modifications to these schemes can be made and will be suggested to one skilled in the art having referred to this disclosure. The starting materials and the intermediates of the reactions may be isolated and purified if desired using conventional techniques, including but not limited to filtration, distillation, crystallisation, chromatography and the like. Such materials may be characterised using conventional means, including physical constants and spectral data.

Compounds of general formula I or Ia in which R₁ is hydrogen may be obtained by reductive amination between a ketone or an aldehyde of general formula II and an amine of general formula III. The reaction between ketone or aldehyde II and amine III may be carried out either by one-pot reductive amination or with isolation of the imine followed by reduction.

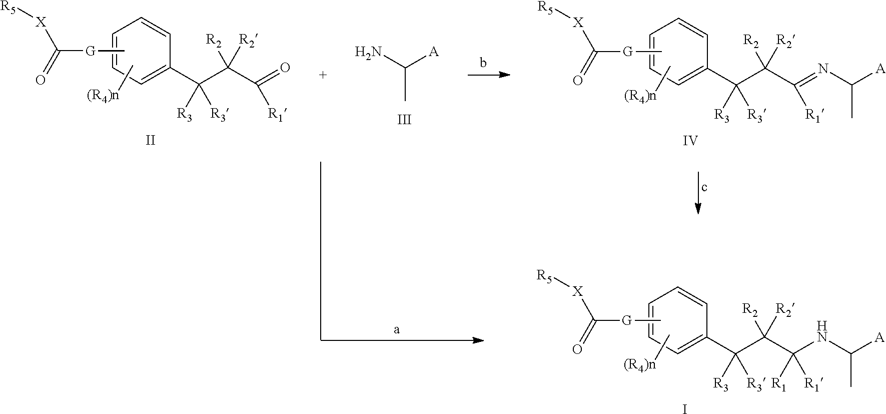

a. The formation of the intermediate iminium IV may be promoted by addition of a protic or aprotic acid such as, but not limited to acetic acid and Ti(Oi-Pr)₄ respectively. The reducing agent may be but is not limited to Na(CN)BH₃, NaBH₄, Na(OAc)₃BH (for other non-limiting conditions see Org. React. 2002, 59, 1-714 and references cited therein).
b. The formation of the imine is promoted either by Lewis acids such as TiCl₄, ZnCl₂, AlCl₃ or by bases such as pyridine, optionally in the presence of a drying agent such as TiCl₄ or molecular sieve (see Comprehensive Organic Functionnal Group Transformations 3, 403 (1995) Pergamon).
c. Reduction may be performed by hydrogenation in the presence of a catalyst such as Pd/C, Pt/C or a chiral rhodium complex to perform the reaction in a stereoselective manner or by hydride transfer from a reducing agent such as BH₃, NaBH₄, NaBH₃CN, LiAlH₄, L-selectride (see Larock R. C. Comprehensive Organic Transformations 1989, VCH; Comprehensive Organic Functionnal Group Transformations 2, 268-269 (2005) Pergamon and references cited therein).

Compounds of general formula I or Ia may also be prepared through alkylation of the amine III.

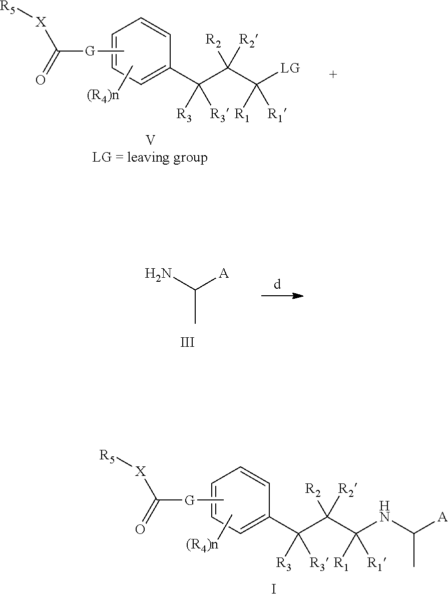

d. When LG is a leaving group such as chloride, bromide, iodide, tosylate or triflate, alkylation is performed in the presence of a base such as NEt₃, DIPEA, NaH, NaOH, KOH, carbonates in an appropriate solvent such as DMF, pyridine, DMSO, CH₃CN, acetone, toluene. Alternatively reaction with an alcohol (LG=OH) may also be considered. Such Mitsunobu-like reaction is performed in the presence of a phosphine such as PBu₃, PPh₃ and the like, an azodicarboxylate or an azodicarboxamide in an aprotic solvent, typically THF. For this purpose the amine III is protected/activated as a carbamate or a sulphonamide. The resulting compound is deprotected using standard conditions (Protective Groups in Organic Synthesis, T. W. Greene and P. G. M. Wuts, John Wiley and Sons, 3^{rd} Edition 1999 and reference sited therein) to afford I or Ia.

The aldehyde or ketone II may be prepared in various manners:

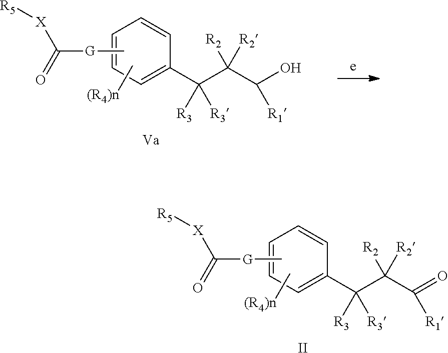

e. An alcohol Va may be oxidised to afford II. Oxidation may be performed with many different reagents. A few of them are H₂Cr₂O₂, Al₂O₃, MnO₂, periodinanes, DMSO in combination with DCC, acetic anhydride, oxalyl chloride and the like.

Alkenones may be used as starting materials.

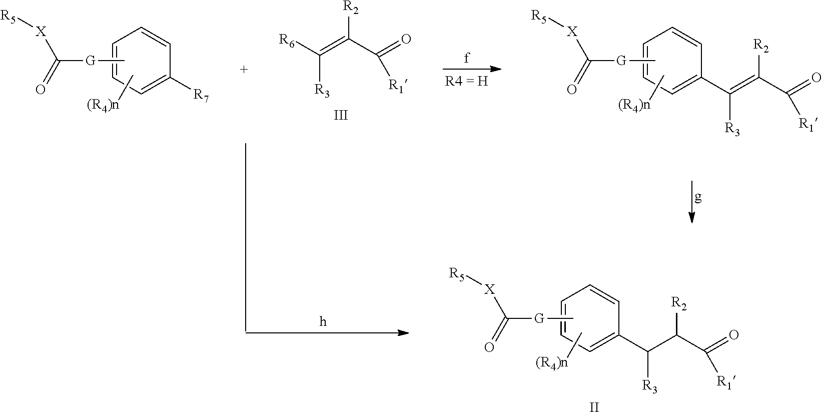

f. Coupling reaction with an aryl halide or pseudo halide such as triflate in the presence of a palladium source such as Pd(OAc)₂, PdCl₂(PPh₃)₂, a base such as NEt₃, K₂CO₃, NaHCO₃, optionally with a phosphine such PPh₃, P(o-Tol)₃, 1,3-bis(diphenylphosphino)propane (dppp), optionally in the presence of a salt like NBu₄Cl, AgNO₃ in a solvent such as DMF or acetonitrile. Alternatively a decarboxylative Heck-type coupling may be performed using an aryl/heteroaryl carboxylic acid (Org. Lett. 2004, 6, 433).
g. Chemospecific reduction of the double bond may be performed under numerous conditions. The hydrogen source may be H₂, water, Hantzsch esters. Metal-based catalysts such as Pd/C, Pd(PPh₃)₄, supported PdCl₂, Rh-, Co-, Cu-, Ir-based catalysts may be used. Stereoselectivity may be achieved by addition of a chiral auxiliary such as but not limited to enantiopure binaphtol phosphate derivatives/valine, imidazolidinone iminiums, bidentate phosphines.

Alternatively alkenones may be subjected to 1,4-addition.
h. Reaction with an aryl metal in which the metal may be Li, Mg halide, trialkyltin, boronic acid, boronic acid ester optionally in the presence of a metal complex such as PdCl₂, Pd(OAc)₂, Pd(PPh₃)₄, (acac)Rh(CO)₂, Ni(acac)₂, (COD)Rh(1,4-dihydroquinone)BF₄ with a ligand typically phosphine-based such as PBu₃, PPh₃, 1,3-bis(diphenylphosphino)propane (dppp), 1,3-hydroquinone or 1,4-hydroquinone in solvents such as DMF, THF, water, toluene, dioxane, dimethoxyethane. In the presence of a chiral ligand as a pure enantiomer such as BINAP, phosphoramidite, Me-DuPHOS and the like the reaction may be performed stereoselectively.

Carboxylates are starting materials for various compounds of general formula I or Ia where the carboxylic group is transformed into amides and esters. Some non-limiting examples are depicted below.

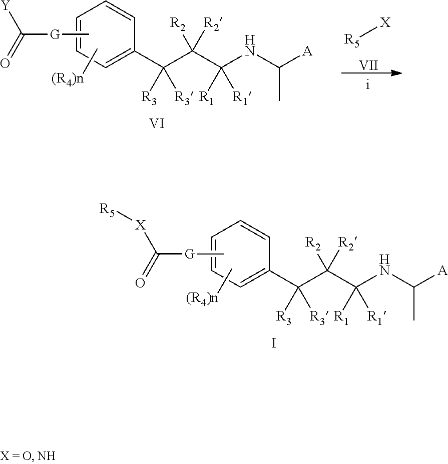

i. The amide or ester may be formed using an amine (VII, X═NH) or an alcohol (VII, X═O) and a carboxylic acid VI (Y═OH) in the presence of coupling agents such as 1,1′-carbonyldiimidazole (CDI), diphenylphosphinic chloride (DPP-Cl), benzotriazol-yloxy-tripyrrolidinophosphonium hexafluorophosphate (PyBOP), benzotriazol-1-yloxy-tris(dimethylamino)-phosphonium hexafluorophosphate (BOP), pentafluorophenyl diphenylphosphinate (fdpp), N-[(dimethylamino)-1H-1,2,3-triazolo[4,5-b]pyridine-1-ylmethylene]-N-methylmethanaminium hexafluorophosphate N-oxide (HATU), bromotripyrrolidinophosphonium hexafluorophosphate (PyBroP), N,N′-dicyclohexylcarbodiimide (DCC), or 1-ethyl-3-(3-dimethylaminopropyl)-carbodiimide; hydrochloride (EDCI). The reaction may be performed in solvents such as diethylether, dichloromethane, 1,2-dichloro-ethane, tetrahydrofuran, 1-methyl-2-pyrrolidinone, dimethylsulfoxyde or dimethylformamide. The reactions are generally carried out in the presence of a base such as Et₃N, DIPEA, collidine or Bu₃N and preferably in the presence of an activator such as HOBt (for example where HOBt is used to improve reactions rates, see Windridge, G. C.; Jorgensen, E. C. JACS 1971, 93, 6318) or HOAt. The amide or ester may also be formed through the reaction of the amine (VII, X═NH) or an alcohol (VII, X═O) and a carboxylic acid chloride (VI, Y═Cl). The carboxylic acid chloride may be prepared by reaction of the carboxylic acid (VI, Y ═OH) with chlorinating agents such as thionyl chloride or oxalyl chloride.

Compounds of general formula I where R2, R2′, R3 and R3′ are hydrogen may be obtained by Sonogashira coupling of a propargylic amine with a suitably substituted iodobenzene, followed by hydrogenation of the product.

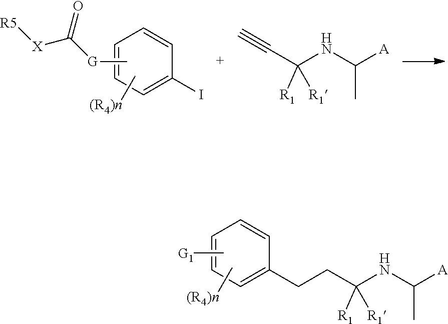

This Sonogashira coupling may be carried out in a solvent like Dioxane, Diethylamine or THF at room temperature or with heating at 50° C. Triethylamine may be used as a base, if the solvent is not itself a basic amine. Suitable catalyst pairs are copper(I) iodide with Palladium(II)(bis-triphenylphosphine) chloride or a similar Palladium(II) or Palladium(0) complex.

The hydrogenation can be effected over a Palladium(0) catalyst, like Palladium on carbon. As a hydrogen source, hydrogen gas may be used. Alternatively, catalytic transfer hydrogenation may be used with hydrogen sources such as cyclohexadiene, ammonium formiate or triethyl silane.

The propargylic amine required for Sonogashira coupling can be prepared by simple alkylation of 1-arylethylamines with propargyl bromide in dimethylformamide and potassium carbonate as a base.

Methods of Preparation (Hydroxylated Chains from Epoxide Opening, and Diverse Substitutents on the Chain from Cross Metathesis Reactions).

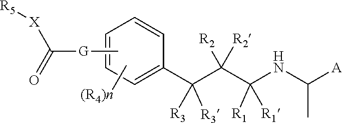

General formula 1.

Compounds of general formula I or Ia with R₂ or R₂′ being hydroxy may be obtained by ring opening of epoxides of general formula F1 with amines of general formula III (Scheme 1).

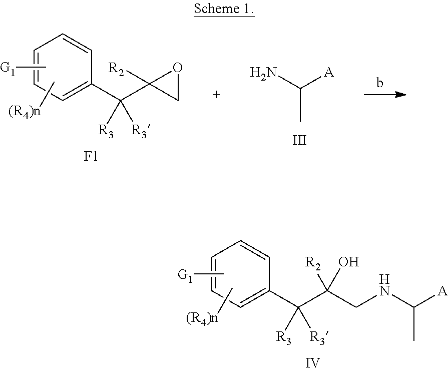

This ring opening may be carried out in a solvent like dioxane, dimethylsulphoxide or dimethylformamide with heating and optional addition of a lithium salt, such as lithium perchlorate, lithium chloride, or another Lewis acid.

When R3 and R3′ are both hydrogen, the required epoxides of general formula F1 may be obtained by opening of an epoxide with an aromatic which has been metallated with such metals as lithium (shown) or magnesium via metal halogen exchange using alkyl metals (Scheme 2).

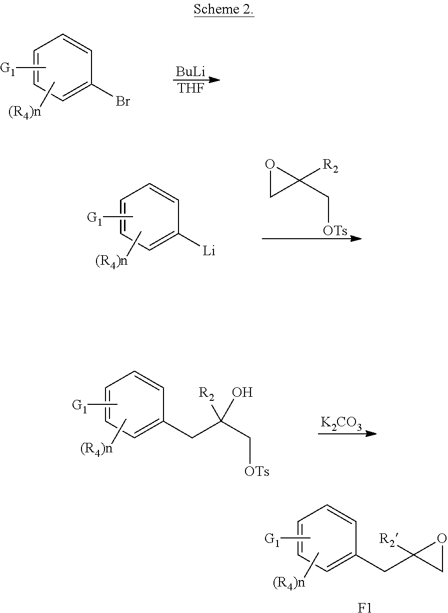

The ring opening reaction is catalyzed by certain Lewis acids such as boron trifluoride, allowing the transformation to be carried out at low temperature (eg.-70° C.) in order to minimize side reactions. Depending on the nature of the group G, the atom X, and the residue R5, this ring opening of epoxide may be carried out at one of several stages of the synthesis:

The epoxide may advantageously be opened while R5 is already in place on the atom X.

Alternatively, the residue R5 may be introduced after opening of an epoxide with the atom X already in place.

Also, both the residue R5 and the atom X may be introduced after opening of an epoxide with the carbonyl group already in place.

Furthermore, the residue R5, the atom X, and the carbonyl group between the atom X and the group G may all be introduced after opening of an epoxide with the group G already in place.

Finally, the epoxide opening may be carried out before the group G has been installed on the phenyl ring.

The resulting hydroxytosylate may then be cyclized with base, such as 2 eq. potassium carbonate in a solvent such as methanol, to form the epoxide ring required (Scheme 2).

Epoxides of general formula F1 may also be prepared by direct epoxidation of alkenes (Scheme 3) using organic peracids like meta-chloroperbenzoic acid in a solvent such as dichloromethane.

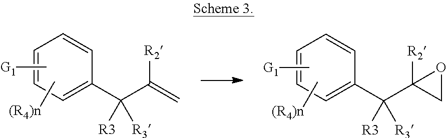

Epoxidation can also be effected in a three step fashion. (Scheme 4). First, an alkene is dihydroxylated under the conditions known as Sharpless' dihydroxylation, using 1,4-Bis(9-O-dihydroquinidinyl)phthalazine, osmium tetraoxide, potassium hexacyano(III)ferrate and potassium carbonate in a mixture of water and tert-butanol or another suitable solvent.

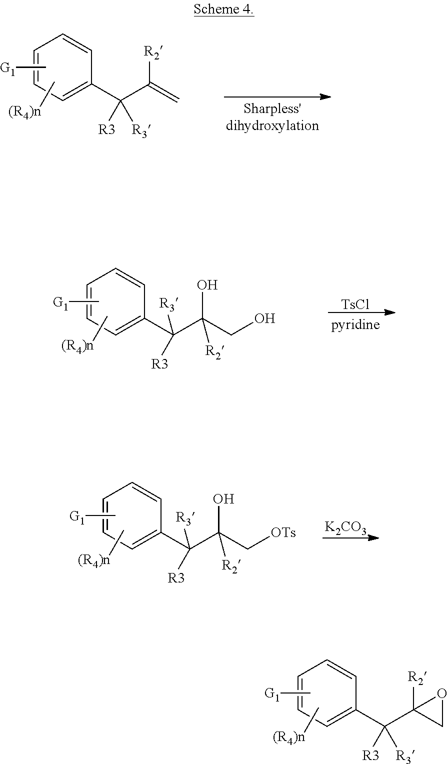

Second, the diol formed is tosylated on the primary hydroxyl with tosyl chloride in pyridine, and finally the epoxide ring is formed upon ring closing of the hydroxyl tosylate with weak base, such as potassium carbonate in acetone or methanol.

If R3 (or R3′) is hydroxyl, epoxidation may be effected using dry tert-butyl hydroperoxide, titanium tetraisopropoxide, and diethyl tartrate in dry dichloromethane (Sharpless' epoxidation, review: A. Pfenninger, Synthesis, 1986 p89-116). Another route to these epoxides involves addition of a lithiated oxirane bearing an electron-withdrawing substituent, R3, to a phenyl ketone or aldehyde (Scheme 5, see Yamauchi, Yoshihiro; Kawate, Tomomi; Katagiri, Toshimasa; Uneyama, Kenji. Tetrahedron (2003), 59 (49), 9839-9847.

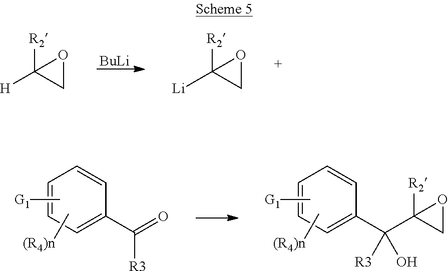

Liu, Bingcan; Das, Sanjoy K.; Roy, Rene. Efficient Ruthenium Carbenoid-Catalyzed Cross-Metathesis of Allyl Halides with Olefins. Organic Letters (2002), 4(16), 2723-2726.

Chiral amines of the general formula III are commercially available or may be prepared from the more readily available aldehydes by catalytic asymmetric synthesis using tert-butanesulfinamide according to Liu, G.; Cogan, D. A.; Ellmann, J. A., J. Amer. Chem. Soc., 1997, 114, 9913.

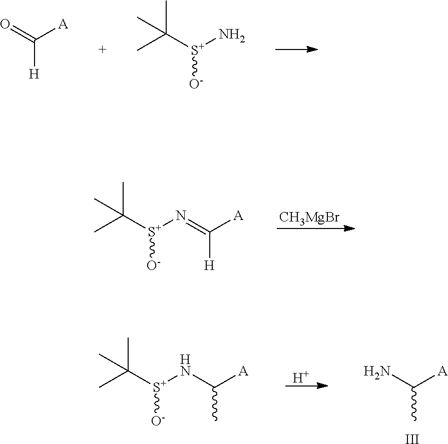

### Pharmaceutical Compositions

For use in therapy, compounds of the present invention are typically in the form of a pharmaceutical composition. The invention therefore relates to a pharmaceutical composition comprising a compound of formula I or Ia, optionally together with one or more other therapeutically active compound(s), together with a pharmaceutically acceptable excipient or vehicle. The excipient must be “acceptable” in the sense of being compatible with the other ingredients of the composition and not deleterious to the recipient thereof.

Conveniently, the active ingredient comprises from 0.05-99.9% by weight of the formulation.

Pharmaceutical compositions of the invention may be in unit dosage form such as tablets, pills, capsules, powders, granules, elixirs, syrups, emulsions, ampoules, suppositories or parenteral solutions or suspensions; for oral, parenteral, opthalmic, transdermal, intra-articular, topical, pulmonal, nasal, buccal or rectal administration or in any other manner appropriate for the formulation of compounds used in nephrology and in accordance with accepted practices such as those disclosed in Remington: The Science and Practice of Pharmacy, 21^{st} ed., 2000, Lippincott Williams & Wilkins. In the composition of the invention, the active component may be present in an amount of from about 0.01 to about 99%, such as 0.1% to about 10% by weight of the composition.

For oral administration in the form of a tablet or capsule, a compound of formula I or Ia may suitably be combined with an oral, non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methyl cellulose, agar, bentonite, xanthan gum or the like. Additional excipients for capsules include macrogols or lipids.

For the preparation of solid compositions such as tablets, the active compound of formula I or Ia is mixed with one or more excipients, such as the ones described above, and other pharmaceutical diluents such as water to make a solid preformulation composition containing a homogenous mixture of a compound of formula I or Ia. The term “homogenous” is understood to mean that the compound of formula I or Ia is dispersed evenly throughout the composition so that the composition may readily be subdivided into equally effective unit dosage forms such as tablets or capsules. The preformulation composition may then be subdivided into unit dosage forms containing from about 0.05 to about 1000 mg, in particular from about 0.1 to about 500 mg, e.g. 10-200 mg, such as 30-180 mg, such as 20-50 mg of the active compound of the invention.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.1 mg and 1000 mg, preferably between 1 mg and 100 mg, such as 5-50 mg of a compound of formula I or Ia.

A suitable dosage of the compound of the invention will depend, inter alia, on the age and condition of the patient, the severity of the disease to be treated and other factors well known to the practising physician. The compound may be administered either orally, parenterally or topically according to different dosing schedules, e.g. daily or with weekly intervals. In general a single dose will be in the range from 0.01 to 400 mg/kg body weight. The compound may be administered as a bolus (i.e. the entire daily dosis is administered at once) or in divided doses two or more times a day.

If the treatment involves administration of another therapeutically active compound it is recommended to consult Goodman & Gilman's The Pharmacological Basis of Therapeutics, 9^{th} Ed., J. G. Hardman and L. E. Limbird (Eds.), McGraw-Hill 1995, for useful dosages of said compounds. The administration of a compound of the present invention with one or more other active compounds may be either concomitantly or sequentially.

Liquid formulations for either oral or parenteral administration of the compound of the invention include, e.g., aqueous solutions, syrups, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrolidone.

For parenteral administration, e.g. intramuscular, intraperitoneal, subcutaneous or intravenous injection or infusion, the pharmaceutical composition preferably comprises a compound of formula I or Ia dissolved or solubilised in an appropriate, pharmaceutically acceptable solvent. For parenteral administration, the composition of the invention may include a sterile aqueous or non-aqueous solvent, in particular water, isotonic saline, isotonic glucose solution, buffer solution or other solvent conventionally used for parenteral administration of therapeutically active substances. The composition may be sterilised by, for instance, filtration through a bacteria-retaining filter, addition of a sterilising agent to the composition, irradiation of the composition, or heating the composition. Alternatively, the compound of the invention may be provided as a sterile, solid preparation, e.g. a freeze-dried powder, which is dissolved in sterile solvent immediately prior to use.

The composition intended for parenteral administration may additionally comprise conventional additives such as stabilisers, buffers or preservatives, e.g. antioxidants such as methyl hydroxybenzoate or the like.

Compositions for rectal administration may be in the form of a suppository incorporating the active ingredient and a carrier such as cocoa butter, or in the form of an enema.

Compositions suitable for intra-articular administration may be in the form of a sterile aqueous preparation of the active ingredient which may be in microcrystalline form, for example, in the form of an aqueous microcrystalline suspension. Liposomal formulations or biodegradable polymer systems may also be used to present the active ingredient for both intra-articular and ophthalmic administration.

Compositions suitable for topical administration, including ophthalmic treatment, include liquid or semi-liquid preparations such as liniments, lotions, gels, applicants, oil-in-water or water-in-oil emulsions such as creams, ointments or pastes; or solutions or suspensions such as drops. For topical administration, the compound of formula I or Ia may typically be present in an amount of from 0.01 to 20% by weight of the composition, such as 0.1% to about 10%, but may also be present in an amount of up to about 50% of the composition. Compositions for ophthalmic treatment may preferably additionally contain a cyclodextrin. Compositions suitable for administration to the nasal or buccal cavity or for inhalation include powder, self-propelling and spray formulations, such as aerosols and atomizers. Such compositions may comprise a compound of formula I or Ia in an amount of 0.01-20%, e.g. 2%, by weight of the composition.

The composition may additionally comprise one or more other active components conventionally used in the treatment of physiological disorders or diseases associated with disturbances of CaSR activity, such as hyperparathyroidism.

A suitable dosage of the compound of the invention will depend, inter alia, on the age and condition of the patient, the severity of the disease to be treated and other factors well known to the practising physician. The compound may be administered either orally, parenterally or topically according to different dosing schedules, e.g. daily or with weekly intervals. In general a single dose will be in the range from 0.01 to 400 mg/kg body weight. The compound may be administered as a bolus (i.e. the entire daily dosage is administered at once) or in divided doses two or more times a day.

### Pharmacological Methods

The calcium sensing receptor (CaSR) and its use in identifying or screening for calcimimetic compounds has been described in EP 637 237, EP 1 296 142, EP 1 100 826, EP 1 335 978, and EP 1 594 446.

In vitro and in vivo methods for testing the compounds of the present invention are well established and may be found in the references listed above, or e.g. in Journal of Biological Chemistry (2004), 279(8), 7254-7263 or in U.S. Pat. No. 5,858,684 and references cited therein.

### Biological Assay for Analysis of In Vitro Activity

The assay investigates a compound's functional ability to act as a biological positive modulator on the human CaSR. Activation of the receptor expressed on CHO-K1 cells is detected through the G alpha q pathway, the activation of phospholipase C and the accumulation of intracellular inositol phosphate (IP) as described earlier [Sandrine Ferry, Bruno Chatel, Robert H. Dodd, Christine Lair, Danielle Gully, Jean-Pierre Maffrand, and Martial Ruat. Effects of Divalent Cations and of a Calcimimetic on Adrenocorticotropic Hormone Release in Pituitary Tumor Cells. BIOCHEMICAL AND BIOPHYSICAL RESEARCH COMMUNICATIONS 238, 866-873 (1997)]. The human CaSR is stably expressed on a CHO-K1 cell clone, stimulated with a basal level of calcium and challenged with the tested compound. The level of IP1 is determined using the IP-One htrf kit (Cisbio, France). CHO-K1 cells not transfected with the CaSR fail to elicit an IP1 response upon calcium and/or compound stimulation.

### Cloning of the Human CaSR Gene

The ORF coding for the human CaSR (genebank: NM_{—}000388) was acquired from Invitrogen Corp, USA and subsequently cloned into the mammalian expression vector pCDA3.1.

### Generation of Cell Line Expressing CaSR

CHO-K1 cells were transfected using Lipofectamine according to manufacturer's protocol (400.000 cells/well were seeded in a 6-well plate and transfected after 24 hours using 2 μg DNA and 5 μl lipofectamine). After another 24 hours the cells were detached, seeded and subjected to 1 mg/ml of G-418. Following 7 days growth single clones were picked, the CaSR expression evaluated using the 5C10 antibody against CaSR, the clones with the highest expression were selected and tested for functional response. The preferred clone was continuously cultured according to standard procedures described in ATCC (American Type Culture Collection) protocols for CHO-K1 with the addition of 500 μg/ml G-418.

### Functional Whole Cell Assay

On the assay day cells were harvested and resuspended to 13*10⁶ cells/ml in stimulation buffer (containing: Hepes 10 mM, MgCl₂ 0.5 mM, KCl 4.2 mM, NaCl-146 mM, glucose 5.5 mM, LiCl 50 mM at pH 7.4). Five μl cell solution were pipetted into a well (white 384-well plate, Perkin Elmer Optiplate) followed by 5 μl compound diluted in a Ca²⁺-containing (to the final concentration of 2 mM) buffer. After compound stimulation for 1 hour at 37° C. 10 ul of IP-One assay reagents were added and incubated for another 1 hour at room temperature. Finally the plate was read using a Perkin Elmer EnVision, according to protocol supplied by the IP-One assay kit manufacturer. The FRET ratio was calculated by dividing the 665 nm emission signal with that of the 615 nm.

Testing data of compounds of the present invention indicate that compounds of the present invention are potent modulators of CaSR, thus making them potentially useful in the treatment of diseases related to kidneys or bones.

As described above, the compounds described in the present invention are modulators of CaSR activity. The CaSR can be found in the parathyroid gland, the thyroid, bone cells, the stomach, the lung, the kidney, pituitary gland, the brain, the hypothalamus, the olfactory areas or the hippocampus. Compounds according to the present invention may preferably be more selective, in their use, with respect to the receptors of the parathyroid compared with those of the thyroid gland.

The compounds according to the invention, and the pharmaceutical compositions comprising them, may be used as a medicinal product, in particular for the treatment of physiological disorders or diseases associated with disturbances of CaSR activity. Even more particularly, these physiological disorders or diseases of the type including primary or secondary hyperparathyroidism, osteoporosis, cardiovascular, gastrointestinal, endocrine or neurodegenerative diseases or certain cancers in which (Ca²⁺)ₑ ions are abnormally high. The secondary hyperparathyroidism is more particularly observed in chronic renal failure.

### Screening for P450 2D6 Inhibition

The assay rapidly screen for potential inhibitors of human P450 2D6 catalytic activity, by using recombinant human P450 2D6. The IC50 determination is performed in duplicate at eight concentrations.

Incubations were conducted in 96 well microtiter plates based on a method described by BD Biosciences. To the first well in each row, a NADPH regenerating system and test compound was added. In the second well and all remaining wells, NADPH regenerating system and acetonitrile (final concentration of 2%) was added. The final assay concentration of the NADPH regenerating system was 8.2 μM NADP⁺, 0.41 mM glucose-6-phosphate, 0.41 mM magnesium chloride hexahydrate and 0.4 U/ml glucose-6-phosphate dehydrogenase and 0.01 mg/mL control insect cell membrane protein. The test compound solution was serially diluted 1:3 through the eighth wells. The final concentration of the test compounds were in the range 100 μM to 45.7 nM in the eight rows. Wells 9 and 10 contained no test compound (only NADPH regenerating system and enzyme/substrate mix) and wells 11 and 12 were used as controls for background fluorescence (enzyme and substrate were added after the reaction was terminated). The plate was then pre-incubated at 37° C. for 10 min, and the reaction was initiated by the addition of pre-warmed enzyme/substrate mix. The assay concentration of the enzyme/substrate mix was 100 mM potassium phosphate, pH 7.4, 1.5 pmol recombinant human P450 CYP2D6 and 1.5 μM of the fluorescent substrate 3-[2-(N,N diethyl-N-methylamino)ethyl]-7-methoxy-4-methylcoumarin (AMMC). The assay was conducted in duplicate in a final volume of 200 μL per well. Reactions were terminated after 30 min by addition of a 4:1, acetonitrile:0.5 M Tris base solution. Quinidine was used as positive control, 0.5 μM as highest concentration. Fluorescence per well was measured using a fluorescence plate reader (excitation: 390 nm, emission: 460 nm). The IC50 values were calculated.

Testing data of compounds of the present invention indicate that compounds of the present invention show low or no inhibition towards human P450 2D6 (pIC50-value below 6).

The invention is described in further detail in the following non-limiting examples which are not in any way intended to limit the scope of the invention as claimed.

### EXAMPLES

### General

All the starting materials used are commercially available, unless otherwise described.

For ¹H nuclear magnetic resonance (NMR) spectra (300 MHz) and ¹³C NMR (75.6 MHz) chemical shift values (δ) (in ppm) are quoted, unless otherwise specified; for deuteriochloroform solutions relative to internal tetramethylsilane (δ=0.00) or chloroform (δ=7.26) or deuteriochloroform (δ=76.81 for ¹³C NMR) standard. The value of a multiplet, either defined (doublet (d), triplet (t), quartet (q), pentet (p), doublet of doublets (dd), doublet of triplets (dt)) or not (m) at the approximate mid point is given unless a range is quoted. All organic solvents used were anhydrous.

For some of the compounds, only LC/MS data are given. Two methods for LC/MS analysis are used:

### Method A

Analytical HPLC/MS was performed on a Dionex APS-system with a P680A analytical pump and a Thermo MSQ Plus mass spectrometer. Column: Waters XTerra C-18, 150 mm×4.6 mm, 5 μm; solvent system: A=water (0.1% formic acid) and B=acetonitrile (0.1% formic acid); flow rate=1.0 mL/min; method (10 min): Linear gradient method going from 10% B to 100% in 6.6 minutes and staying at 100% B for another 1.5 minutes.

### Method B

Analytical UPLC/MS was performed on a Waters Acquity UPLC system with a Waters LCT Premier XE mass spectrometer. Column: Waters Acquity UPLC HSS T3 1.8 5 μm; solventsystem: A=water (0.1% formic acid) and B=acetonitrile (0.1% formic acid); Method: 95% A and 5% B with a flow rate=0.350 mL/min for 0.5 minutes; Linear gradient going from 95% A and 5% B to 5% A and 95% B in 2.5 minutes and staying at this level for another 1.5 minutes with a flow rate=0.350 mL/min; Linear gradient going from 5% A and 95% B to 95% A and 5% B in 1 minutes with a flow rate=0.700 mL/min and then decreasing the flow rate to 0.35 mL/min during 0.8 minutes.

Flash chromatography was performed on silica gel. Appropriate mixtures of ethyl acetate, dichloromethane, methanol, and petroleum ether (40-60) were used as eluents unless otherwise noted.

HPLC purifications of the crude products were performed by using Waters LC-MS system [column: Waters X Terra C18, 5 μm or Luna C18 100 Å 5μ; Size: 250×10.00 mm (Phenomenex)]; Sample Manager: Waters 2767; Pump: Waters 2525; Single Quadrupole Waters ZQ; PDA-detector: Waters 2996).

The following abbreviations have been used:

| | |
|---|---|
| aq. | aqueous |
| DAST | (Diethylamino)sulphur trifluoride |
| 1,2-DCE | 1,2-Dichloroethane |
| DCM | Dichloromethane |
| DIPEA | Diisopropylethylamine |
| DMAP | 4-Dimethylaminopyridine |
| DMF | N,N-Dimethylformamide |
| DMSO | Dimethylsulphoxide |
| Et₂O | Diethyl ether |
| EtOAc | Ethyl acetate |
| h | Hour(s) |
| HATU | O-(7-Azabenzotriazol-1-yl)-N,N,N′,N′- |
| | tetramethyluronium hexafluorophosphate |
| HPLC | High performance liquid chromatography |
| min | Minutes |
| NMR | Nuclear magnetic resonance |
| PE | Petroleum ether |
| rt | Room temperature |
| TEA | Triethylamine |
| TFA | Trifluoroacetic acid |
| THF | Tetrahydrofuran |

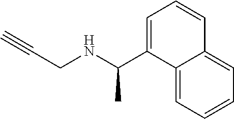

### Preparation 1

(R)-(1-Naphthalen-1-yl-ethyl)-prop-2-ynyl-amine.

To a solution of (R)-1-naphthalen-1-yl-ethylamine (7.1 ml, 44 mmol) in DMSO (100 ml) was added Cs₂CO₃ followed by dropwise addition of propargyl bromide with vigorous stirring. After complete addition, the reaction mixture was stirred for 2 hours at rt and filtered. Brine, water and diethyl ether were added. The aqueous phase was separated and extracted three times with diethyl ether. The combined organic extracts were dried over MgSO₄ and concentrated under reduced pressure to afford a yellow-brown oil. Chromatography afforded the title compound as a light yellow oil. ¹³C NMR (75 MHz, DMSO) δ 140.47, 133.49, 130.85, 128.61, 126.81, 125.68, 125.58, 125.24, 122.91, 82.85, 73.55, 51.18, 35.21, 23.21.

General procedure A (Sonogashira)

(R)-(1-Naphthalen-1-yl-ethyl)-prop-2-ynyl-amine (preparation 1) (1.4 mmol, 300 mg) and aryl iodide (1.4 mmol) were dissolved in 3 ml diethylamine in an 8 ml vial. CuI (0.09 mmol, 0.06 eq.) and Pd(PPh₃)₂Cl₂ (0.03 mmol, 0.02 eq.) were added. The vial was sealed with a screw cap and shaken overnight at r.t. The reaction mixture was diluted with ethyl acetate (10 ml), and the precipitate was filtered off. The filtrate was concentrated under reduced pressure and purified by chromatography.

General Procedure B (hydrogenation)

A solution of alkyne (0.75 mmol) in methanol (5 ml) containing Pd/C (20 mg) was hydrogenated for 1.5 hours. The catalyst was filtered off through Celite, and the filtrate was concentrated under reduced pressure to afford the hydrogenated product.

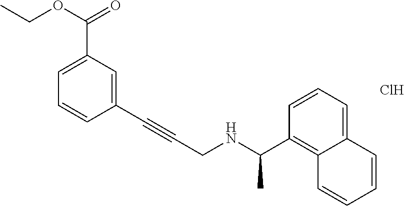

### Preparation 2

Ethyl(R)-3-[3-(1-naphthalen-1-yl-ethylamino)-prop-1-ynyl]-benzoate hydrochloride.

General procedure A was followed using 3-iodo-benzoic acid ethyl ester as the aryl iodide. Chromatography using 0-25% ethyl acetate in heptane afforded the title compound as a neutral compound. The neutral compound was dissolved in ethyl acetate and treated with 4N HCl in dioxane. Precipitation occurred upon addition of diethyl ether. The precipitate was filtered off and dried in vacuo to afford the title compound. NMR of neutral compound: ¹³C NMR (75 MHz, DMSO) δ 164.89, 140.47, 135.46, 133.48, 131.50, 130.89, 130.23, 129.15, 128.62, 128.58, 126.83, 125.63, 125.59, 125.23, 123.19, 123.08, 122.89, 90.23, 81.69, 60.91, 51.27, 36.00, 23.31, 13.98.

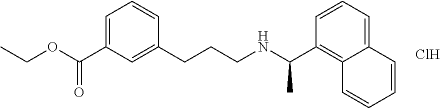

### Example 1

Ethyl(R)-3-(3-{1-naphthalen-1-yl-ethylamino}-propyl)-benzoate, hydrochloride (compound 1001).

General procedure B was followed using ethyl(R)-3-(3-{1-naphthalen-1-yl-ethylamino}-prop-1-ynyl)-benzoate hydrochloride (preparation 2), affording the title compound as an off-white solid. ¹³C NMR (75 MHz, DMSO) δ 166.09, 141.81, 134.54, 133.73, 133.47, 130.66, 130.39, 129.28, 129.15, 127.28, 126.53, 125.90, 124.65, 122.98, 61.05, 52.45, 45.19, 32.00, 27.62, 20.29, 14.55.

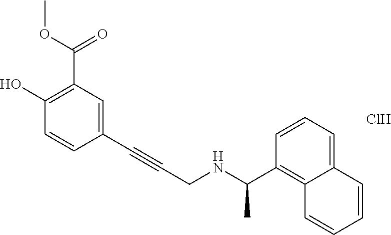

### Preparation 3

Methyl(R)-2-hydroxy-5-[3-(1-naphthalen-1-yl-ethylamino)-prop-1-ynyl]-benzoate hydrochloride.

General procedure A was followed using (R)-2-hydroxy-5-iodo-benzoic acid methyl ester as the aryl iodide. The product was purified by flash chromatography using 0-25% ethyl acetate in heptane and subsequently converted to the title compound by the addition of HCl in dioxane and filtration of the precipitate. ¹³C NMR (75 MHz, DMSO) δ 167.91, 159.20, 140.53, 137.61, 133.47, 133.01, 130.89, 128.61, 126.79, 125.64, 125.59, 125.22, 123.06, 122.89, 117.96, 113.83, 113.63, 87.68, 81.61, 52.45, 51.20, 35.98, 23.33.

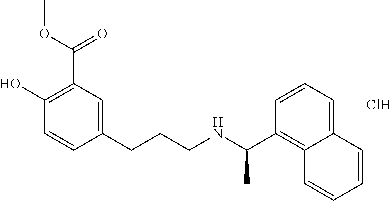

### Example 2

(R)-2-Hydroxy-5-[3-(1-naphthalen-1-ykethylamino)-propyl]-benzoic acid methyl ester hydrochloride (compound 1002). General procedure B was followed using (R)-2-hydroxy-5-[3-(1-naphthalen-1-yl-ethylamino)-prop-1-ynyl]-benzoic acid methyl ester hydrochloride (preparation 3). ¹H NMR (300 MHz, DMSO) δ 10.32 (s, 1H), 9.65 (br, 1H), 9.1 (br, 1H), 8.23 (d, 1H), 8.05-7.88 (m, 3H), 7.7-7.6 (m, 3H), 7.55 (d, 1H), 7.29 (dd, 1H), 6.88 (d, 1H), 5.30 (s, 1H), 3.88 (s, 3H), 2.94 (m, 1H), 2.73 (m, 1H), 1.92 (m, 2H), 1.66 (d, 3H).

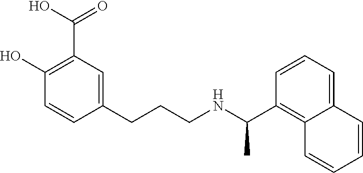

### Example 3

(R)-2-Hydroxy-5-[3-(1-naphthalen-1-yl-ethylamino)-propyl]-benzoic acid (compound 1003).

To a suspension of (R)-2-hydroxy-5-[3-(1-naphthalen-1-yl-ethylamino)-propyl]-benzoic acid methyl ester hydrochloride (compound 1002) (198 mg, 0.57 mmol) in methanol (1.2 ml) and water (0.4 ml) was added LiOH (68 mg). The resulting solution was stirred for 5 days at rt. The solvents were removed in vacuo, and the residue was purified by preparative HPLC to afford the title compound. ¹³C NMR (75 MHz, DMSO) δ 172.24, 160.48, 134.68, 133.29, 131.57, 130.28, 129.40, 128.80, 128.62, 128.29, 126.76, 126.01, 125.52, 123.85, 122.55, 119.61, 115.47, 52.02, 45.18, 31.30, 28.22, 20.09.

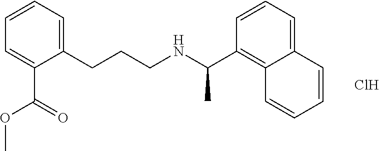

### Example 4

Methyl(R)-2-[3-(1-naphthalen-1-yl-ethylamino)-propyl]-benzoate hydrochloride (compound 1004).

The title compound was prepared in two steps:

Step 1: General procedure A was followed using 2-iodo-benzoic acid methyl ester as the aryl iodide. Chromatography using 0-5% methanol in DCM afforded (R)-2-[3-(1-naphthalen-1-yl-ethylamino)-prop-1-ynyl]-benzoic acid methyl ester. This compound was dissolved in ethyl acetate and treated with HCl in methanol. Upon stirring, a precipitation was formed which was filtered off and dried in vacuo to afford methyl(R)-2-[3-(1-naphthalen-1-yl-ethylamino)-prop-1-ynyl]-benzoate hydrochloride as a yellow solid.

Step 2: General procedure B was followed using (R)-2-[3-(1-naphthalen-1-yl-ethylamino)-prop-1-ynyl]-benzoic acid methyl ester hydrochloride from step 1, affording the title compound. ¹³C NMR (151 MHz, DMSO) δ 167.14, 141.81, 134.05, 133.25, 132.11, 130.73, 130.20, 130.13, 129.07, 128.81, 126.82, 126.37, 126.06, 125.45, 124.25, 122.53, 51.91, 44.89, 39.94, 30.40, 27.31, 19.85.

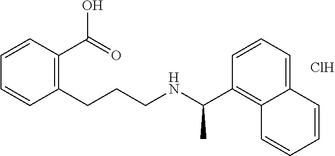

### Example 5

2-[3-((R)-1-Naphthalen-1-yl-ethylamino)-propyl]-benzoic acid hydrochloride (compound 1005). Methyl(R)-2-[3-(1-naphthalen-1-yl-ethylamino)-propyl]-benzoate hydrochloride (compound 1004) (0.20 mmol) was dissolved in methanol (600 μl). Water (200 μl) and LiOH (24 mg) were added, and the mixture was stirred for 5 days at rt. The solvents were removed in vacuo and the residue was purified by HPLC to afford the title compound. ¹³C NMR (75 MHz, DMSO) δ 171.27, 138.54, 138.02, 135.87, 133.24, 130.33, 129.40, 129.00, 128.88, 128.72, 128.10, 126.48, 125.77, 125.55, 125.44, 123.60, 122.53, 51.71, 43.81, 28.61, 27.69, 20.68.

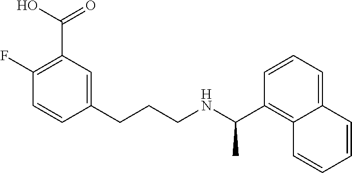

### Example 6

(R)-2-Fluoro-5-[3-(1-naphthalen-1-yl-ethylamino)-propyl]-benzoic acid (compound 1006). The title compound was prepared in three steps:

Step 1: General procedure A was followed using 2-fluoro-5-iodo-benzonitrile as aryl iodide to afford (R)-2-fluoro-5-[3-(1-naphthalen-1-yl-ethylamino)-prop-1-ynyl]-benzonitrile. The product was purified by chromatography using 0-5% methanol in DCM, affording (R)-2-fluoro-5-[3-(1-naphthalen-1-yl-ethylamino)-prop-1-ynyl]-benzonitrile. This compound was converted to the hydrochloride salt by dissolution in ethyl acetate and treatment with 4N HCl in dioxane. Upon the addition of diethyl ether, precipitation occurred. The precipitate was filtered off and dried in vacuo to afford (R)-2-fluoro-5-[3-(1-naphthalen-1-yl-ethylamino)-prop-1-ynyl]-benzonitrile hydrochloride.

Step 2: A solution of (R)-2-fluoro-5-[3-(1-naphthalen-1-yl-ethylamino)-prop-1-ynyl]-benzonitrile hydrochloride (269 mg, 0.82 mmol) in methanol (5 ml) containing Pd/C (10 mg) was hydrogenated by bubbling H₂-gas into the solution for 2 min with stirring. The mixture was filtered through Celite and concentrated under reduced pressure to afford crude (R)-2-fluoro-5-[3-(1-naphthalen-1-yl-ethylamino)-propyl]-benzonitrile hydrochloride, which was used without further purification.

Step 3: (R)-2-Fluoro-5-[3-(1-naphthalen-1-yl-ethylamino)-propyl]-benzonitrile hydrochloride (239 mg, 0.65 mmol) was added to a mixture of NaOH (28% aq., 3 ml) and methanol (7 ml) and was refluxed at 100° C. overnight. The mixture was cooled to r.t., concentrated under reduced pressure to remove methanol, and acidified with aqueous HCl to pH 2. The precipitate thus formed was filtered off and purified by preparative HPLC to afford the title compound as a yellow solid. ¹H NMR (300 MHz, DMSO) δ 8.24 (d, 1H), 7.99-7.93 (m, 1H), 7.84 (dd, 2H), 7.62-7.49 (m, 4H), 7.27-7.19 (m, 1H), 7.05 (dd, 1H), 4.92 (q, 1H), 2.77-2.52 (m, 4H), 1.90-1.75 (m, 2H), 1.52 (d, 3H).

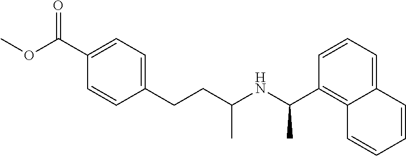

### Example 7

4-[3-((R)-1-Naphthalen-1-yl-ethylamino)-butyl]-benzoic acid methyl ester (compound 1007). To a solution of 4-methoxycarbonylphenylboronic acid (30 mg, 0.2 mmol) and but-3-en-2-one (70 mg, 1 mol) in 0.5 mL DME was added a solution of [(1,4-hydroquinone)-rhodium(COD)]BF₄ (Son et al., J. Am. Chem. Soc. 2005, 127, 12238) (0.67 mg, 0.008 mmol) in 0.5 mL DME and LiOH (0.19 mg, 0.008 mmol) in 1 mL water. The mixture was stirred at 50° C. and solvents were removed under reduced pressure. The residue was redissolved in DCE. (+)-(R)-1-Naphthalen-1-yl-ethylamine (0.2 mmol), NaBH(OAc)₃ (0.24 mmol) and acetic acid (0.28 mmol) were added, and the reaction mixture was stirred overnight at rt. After removing the solvent in vacuo, the residue was redissolved in DMF and purified by preparative HPLC to afford the title compound as a mixture of two isomers. ¹H NMR (300 MHz, DMSO) δ 8.30 (dd, 1H), 7.97-7.89 (m, 1H), 7.84-7.69 (m, 4H), 7.59-7.45 (m, 3H), 7.21 (dd, 2H), 4.86 (q, 1H), 3.83 (d, 3H), 2.79-2.40 (m, 3H), 1.90-1.49 (m, 2H), 1.40 (dd, 3H), 1.06 (dd, 3H).

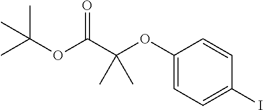

### Preparation 4

tert-Butyl 2-(4-iodophenoxy)-2,2-dimethylacetate.

4-Iodophenol (5 g) was dissolved in DMF, 15 ml, and treated with tert-butyl 2-bromo-2,2-dimethylacetate (3 eq.) and caesium carbonate (4 eq.). After stirring for 18 hours at 75° C., the mixture was cooled and neutralized with 1M HCl in diethyl ether (4 eq.). Solvents were removed in vacuo and the residue purified by chromatography in a gradient of ethyl acetate in hexane (0-50%).

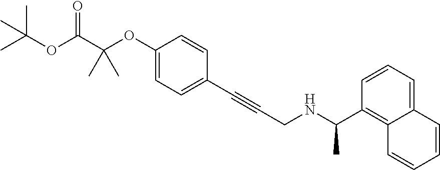

### Preparation 5

tert-Butyl(R)-(2,2-Dimethyl-2-{4-[3-(1-naphthalen-1-yl-ethylamino)-1-propyn-1-yl]-phenoxy}-acetoylamino)-acetate. tert-Butyl 2-(4-iodophenoxy)-2,2-dimethylacetate (preparation 4, 1 g) was dissolved in THF (9 ml) and triethylamine (3 ml), and (R)-(1-naphthalen-1-yl-ethyl)-prop-2-ynyl-amine (preparation 1, 635 mg, 1,1 eq.) was added. Argon was passed through the suspension for a few minutes, and a minimal amount of bis-(Triphenylphosphine)dichloropalladium(II) complex and Copper(I) iodide was added, and stirring maintained for 2 hours at room temperature. Silicagel (5 g) was added and solvents removed in vacuo. Chromatography in a gradient of Ethyl acetate in p. Ether (0-30%) afforded the product.

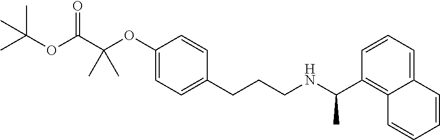

### Example 8

tert-Butyl(R)-(2,2-Dimethyl-2-{4-[3-(1-naphthalen-1-yl-ethylamino)-propyl]-phenoxy}-acetoylamino)-acetate (compound 1008). tert-Butyl(R)-(2,2-dimethyl-2-{4-[3-(1-naphthalen-1-yl-ethylamino)-1-propynyl]-phenoxy}-acetoylamino)-acetate (preparation 5, 600 mg) was dissolved in methanol. A catalytic amount of Palladium on carbon (5%) was added. Hydrogen was supplied from a balloon and the mixture was shaken at room temperature for 28 hours. The suspension was filtered through Celite, silicagel was added to the filtrate and solvents were removed in vacuo. The residue was purified by chromatography in a gradient of ethyl acetate in heptane (0-100%). ¹H NMR (300 MHz, DMSO) δ 8.25 (d, 1H), 7.91 (d, 1H), 7.79 (d, 1H), 7.70 (d, 1H), 7.56-7.41 (m, 3H), 7.00 (dd, 2H), 6.66 (dd, 2H), 4.70-4.51 (m, 1H), 2.48 (t, 4H), 1.80-1.57 (m, 2H), 1.51-1.23 (m, 18H).

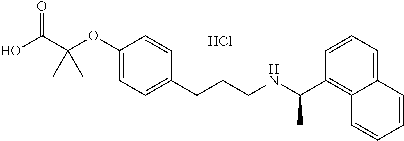

### Example 9

(R)-(2,2-Dimethyl-2-{4-[3-(1-naphthalen-1-yl-ethylamino)-propyl]-phenoxy}-acetoylamino)-acetic acid, hydrochloride (compound 1009). tert-Butyl (R)-(2,2-dimethyl-2-{4-[3-(1-naphthalen-1-yl-ethylamino)-propyl]-phenoxy}-acetoylamino)-acetate (compound 1008, 600 mg) was dissolved in acetonitrile (10 ml). HCl in dioxane (2 ml, 4M) was added, and the solution was stirred for 3 hours at 40° C. The product crystallized spontaneously and was collected on a filter and dried in vacuo. ¹³C NMR (75 MHz, DMSO) δ 174.93, 153.39, 134.04, 133.67, 133.26, 130.22, 128.81, 128.75, 126.84, 126.06, 125.45, 124.30, 122.53, 118.52, 78.21, 51.93, 44.74, 30.95, 27.08, 24.95, 19.86.

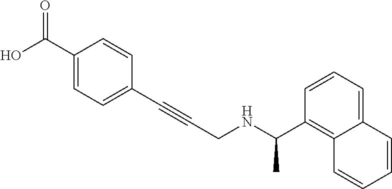

### Preparation 6

(R)-4-[3-(1-Naphthalen-1-yl-ethylamino)-propyn-1-yl]-benzoic acid.

General procedure A was followed using 4-iodobenzoic acid as the aryl iodide. ¹H NMR (300 MHz, DMSO) δ 8.39-8.30 (m, 1H), 7.98-7.86 (m, 3H), 7.81 (d, 1H), 7.72 (d, 1H), 7.59-7.48 (m, 3H), 7.45 (d, 2H), 4.88 (q, 1H), 3.62 (d, 1H), 3.38 (d, 1H), 1.42 (d, 3H).

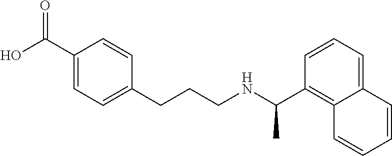

### Example 10

(R)-4-[3-(1-Naphthalen-1-yl-ethylamino)-propyl]-benzoic acid (compound 1010).

General procedure B was followed using (R)-4-[3-(1-naphthalen-1-yl-ethylamino)-propyn-1-yl]-benzoic acid (preparation 6). ¹³C NMR (75 MHz, DMSO) δ 167.13, 145.99, 134.64, 133.27, 130.25, 129.31, 128.80, 128.63, 128.60, 128.26, 126.73, 125.97, 125.46, 124.21, 122.55, 52.08, 44.82, 31.85, 27.02, 20.18.

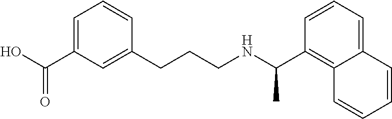

### Example 11

(R)-3-(3-{1-naphthalen-1-yl-ethylamino}-propyl)-benzoic acid (compound 1011). Prepared from ethyl(R)-3-(3-{1-naphthalen-1-yl-ethylamino}-propyl)-benzoate, hydrochloride (compound 1001) by hydrolysis with 2 eq. KOH and 10 eq. water in a 3:1 mixture of methanol and dioxane. After stirring over night at 50° C., solvents were removed in vacuo. The residue was suspended in dioxane and passed through a filter. The filtrate was acidified with 4 N HCl in dioxane and stripped of solvents in vacuo. The residue was purified by HPLC. ¹³C NMR (75 MHz, DMSO) δ 167.61, 163.74, 162.20, 147.28, 141.84, 138.86, 133.37, 132.26, 131.66, 130.61, 128.95, 128.66, 128.24, 127.36, 126.69, 126.05, 125.51, 123.09, 122.80.

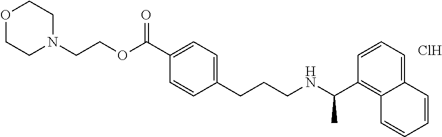

### Example 12

(R)-4-[3-(1-Naphthalen-1-yl-ethylamino)-propyl]-benzoic acid 2-morpholin-4-yl-ethyl ester hydrochloride (compound 1012). To a solution of (R)-4-[3-(1-naphthalen-1-yl-ethylamino)-propyl]-benzoic acid (compound 1010, 650 mg, 1.95 mmol) in DMF (6 ml) under argon was added 1,1′-carbonyldiimidazole (CDI) (379 mg, 2.33 mmol, 1.2 eq.). The resulting mixture was stirred at rt for 4 h. One third of the mixture was transferred to a new reaction flask, and 2-morpholin-4-yl-ethanol (852 mg, 6.4 mmol, 10 eq.) was added. After stirring overnight, the reaction mixture was concentrated under reduced pressure and purified by chromatography (0-20% methanol in dichloromethane). The product was dissolved in ethyl acetate and treated with 4N HCl in dioxane. Precipitation occurred upon addition of diethyl ether. The precipitate was filtered off and dried in vacuo to afford the title compound as a white solid. ¹³C NMR (75 MHz, MeOH) δ 167.27, 148.01, 135.55, 134.20, 132.13, 131.23, 131.02, 130.43, 129.72, 128.71, 128.56, 127.61, 126.64, 124.96, 123.05, 65.02, 59.76, 57.31, 54.28, 53.76, 46.82, 33.49, 28.64, 20.18.

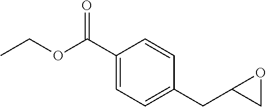

### Preparation 7

Ethyl (±)-4-(oxiranylmethyl)-benzoate.

Ethyl 4-prop-2-enylbenzoate was treated with 1.3 eq. MCPBA in Dichloromethane at room temperature over night and purified by chromatography. The yield was 71%.

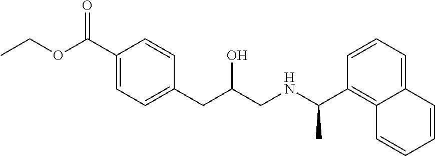

### Example 13

Ethyl 4-(2-hydroxy-3-{(R)-1-naphthalen-1-yl-ethylamino}-propyl)-benzoate (compound 1013). (Mixture of stereoisomers) Ethyl (±)-4-(oxiranylmethyl)-benzoate (preparation 7) was dissolved in acetonitrile, and (+)-(R)-1-naphthalene-1-yl-ethylamine (2 eq.) was added, followed by anhydrous lithium perchlorate (2 eq.). After stirring at 50° C. for 40 hours, solvents were removed and the residue was purified by chromatography yielding the product as a mixture of diastereomers (colourless oil). ¹H NMR (300 MHz, CDCl3) δ 8.18-8.08 (m, 1H), 7.97-7.90 (m, 2H), 7.90-7.83 (m, 1H), 7.76 (d, 1H), 7.60-7.54 (m, 1H), 7.54-7.43 (m, 3H), 7.22 (dd, 2H), 4.65 (p, 1H), 4.35 (q, 2H), 3.97-3.79 (m, 1H), 2.83-2.63 (m, 3H), 2.51 (ddd, 1H), 1.55-1.46 (m, 3H), 1.38 (dd, 3H).

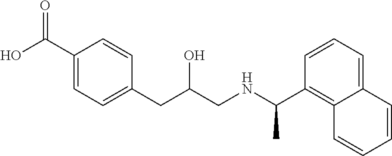

### Example 14

4-(2-hydroxy-3-{(R)-1-naphthalen-1-yl-ethylamino}-propyl)-benzoic acid (compound 1014). (Mixture of stereoisomers)

Ethyl 4-(2-hydroxy-3-{(R)-1-naphthalen-1-yl-ethylamino}-propyl)-benzoate (compound 1013, mixture of stereoisomers) was hydrolyzed with lithium hydroxide (1.4 eq.) in a mixture of methanol and water. At completion, methanol was removed in vacuo, the solution was neutralized by careful addition of 1.4 eq. hydrochloric acid. The title compound precipitated from water as the zwitter-ion. ¹H NMR (300 MHz, DMSO) δ 8.25 (d, 1H), 7.96-7.88 (m, 1H), 7.83-7.75 (m, 3H), 7.72-7.65 (m, 1H), 7.57-7.44 (m, 3H), 7.26 (dd, 2H), 4.59 (q, 1H), 3.87-3.74 (m, 1H), 2.89-2.72 (m, 1H), 2.71-2.56 (m, 1H), 2.55-2.34 (m, 5H).

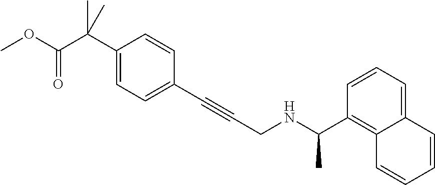

### Preparation 8

Methyl(R)-2′,2′-Dimethyl-2′-{4-[3-(1-naphthalen-1-yl-ethylamino)-propyn-1-yl]-phenyl}-acetate.

General procedure A was followed using methyl 4-iodophenyl-2′,2′-dimethylacetate as the aryl iodide. Purification by chromatography afforded the title compound. ¹³C NMR (75 MHz, DMSO) δ 175.83, 145.76, 133.33, 131.51, 130.16, 129.06, 128.86, 126.81, 126.11, 125.98, 125.50, 124.54, 122.48, 119.27, 86.86, 80.66, 52.06, 51.12, 46.15, 34.69, 26.03, 19.63.

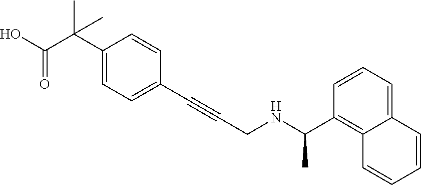

### Preparation 9

(R)-2′,2′-Dimethyl-2′-{4-[3-(1-naphthalen-1-yl-ethylamino)-propyn-1-yl]-phenyl}-acetic acid, hydrochloride. Methyl(R)-2′,2′-dimethyl-2′-{4-[3-(1-naphthalen-1-yl-ethylamino)-1-propyn-1-yl]-phenyl}-acetate (preparation 8) was hydrolyzed with lithium hydroxide in methanol/water. At completion, methanol was removed in vacuo, and after careful neutralization with HCl, the title compound was collected as the hydrochloride. ¹³C NMR (75 MHz, DMSO) 176.98, 146.27, 133.59, 133.34, 131.36, 130.18, 128.99, 128.85, 126.78, 126.06, 125.50, 124.41, 122.48, 119.06, 86.79, 80.87, 51.12, 45.79, 34.80, 26.05, 19.71.

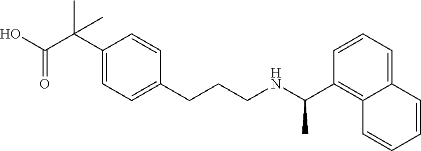

### Example 15

(R)-2′,2′-Dimethyl-2′-{4-[3-(1-naphthalen-1-yl-ethylamino)-propyl]-phenyl}-acetic acid, hydrochloride (compound 1015). General procedure B was followed using (R)-2′,2′-dimethyl-2′-{4-[3-(1-naphthalen-1-yl-ethylamino)-propyn-1-yl]-phenyl}-acetic acid (preparation 9). ¹³C NMR (151 MHz, DMSO) δ 177.54, 142.53, 139.10, 133.31, 130.44, 129.25, 128.72, 128.22, 127.91, 126.37, 125.72, 125.49, 125.39, 123.66, 122.70, 52.35, 45.48, 45.27, 31.70, 28.57, 26.32, 21.29.

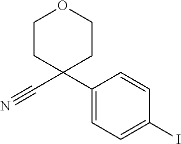

### Preparation 10

4-(4-Iodophenyl)-tetrahydropyrane-4-carbonitrile.

To a solution of (4-iodophenyl)-acetonitrile (2.43 g, 10.0 mmol) in DMSO (50 ml) was added sodium hydride (60% in oil, 1.2 g, 30 mmol) at room temperature. After stirring for 30 min., bis-2-chloroethyl ether (1.57 g, 11 mmol) was added. After stirring for a further 1 hr. at room temperature, the solution was poured into brine/water (1:1) and extracted twice with diethyl ether. The combined organic phases were dried over magnesium sulphate and concentrated in vacuo. The residue was purified by chromatography (15% ethyl acetate in petroleum ether), giving the title compound as a solid.

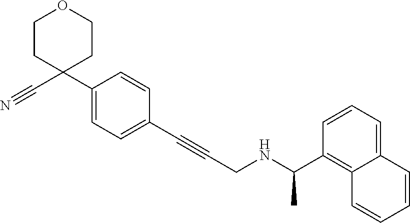

### Preparation 11

(R)-4-(4-[3-(1-naphthalen-1-yl-ethylamino)-prop-1-ynyl]-phenyl)-tetrahydropyrane-4-carbonitrile. General procedure A was followed using 4-(4-iodophenyl)-tetrahydropyrane-4-carbonitrile (preparation 10) as the aryl iodide. The product was purified by chromatography in ethyl acetate and petroleum ether (1:2) giving the title compound as an oil. ¹³C NMR (75 MHz, CDCl3) δ 140.11, 139.47, 134.05, 132.37, 131.40, 129.01, 127.50, 125.84, 125.76, 125.50, 125.39, 123.56, 123.23, 122.99, 121.45, 89.11, 82.66, 64.97, 51.90, 41.82, 36.95, 36.57, 23.14.

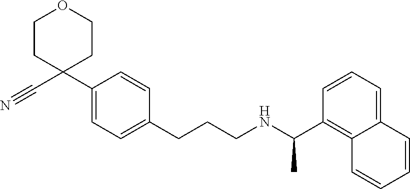

### Preparation 12

(R)-4-(4-[3-(1-naphthalen-1-yl-ethylamino)-propyl]-phenyl)-tetrahydropyrane-4-carbonitrile. General procedure B was followed using (R)-4-(4-[3-(1-naphthalen-1-yl-ethylamino)-prop-1-ynyl]-phenyl)-tetrahydropyrane-4-carbonitrile (preparation 11). ¹³C NMR (75 MHz, DMSO) δ 140.61, 137.60, 134.03, 133.26, 130.22, 128.78, 128.74, 126.83, 126.05, 125.48, 124.32, 122.53, 121.94, 64.27, 51.92, 44.67, 40.54, 35.54, 31.27, 26.82, 19.88.

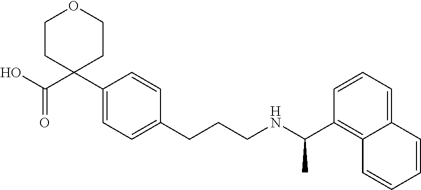

### Example 16

(R)-4-(4-[3-(1-naphthalen-1-yl-ethylamino)-propyl]-phenyl)-tetrahydropyrane-4-carboxylic acid (compound 1016).

(R)-4-(4-[3-(1-naphthalen-1-yl-ethylamino)-propyl]-phenyl)-tetrahydropyrane-4-carbonitrile (preparation 12) was hydrolyzed with a large excess of sodium hydroxide in methanol and water (1:4) at reflux over night. After neutralization with hydrochloric acid and dilution with water, methanol was removed in vacuo. The product precipitated and was collected on a filter and washed with water and diethyl ether, affording, after drying, the title compound as a colourless solid. ¹³C NMR (75 MHz, DMSO) δ 175.58, 141.56, 140.32, 139.93, 133.38, 130.75, 128.59, 127.93, 126.86, 125.79, 125.53, 125.38, 125.28, 122.94, 122.88, 65.03, 52.86, 47.79, 46.41, 34.43, 32.26, 30.57, 23.04.

### General Procedure C

To a solution or a suspension of acid (1 eq.) in DMF (1M) under argon was added CDI (1.5 eq.). The mixture was stirred at r.t. for 6 h before addition of the amine (1.2 eq.). Stirring was continued overnight at r.t. The reaction mixture was diluted with water and extracted with ethyl acetate. The combined organic extracts were washed with aqueous NaHCO₃ and brine, dried over Na₂SO₄ and concentrated under reduced pressure. The residue was purified by continuous gradient flash chromatography.

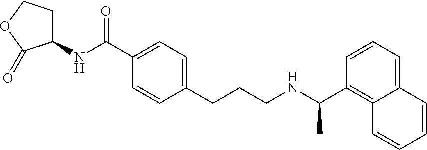

### Example 17

4-[3-((R)-1-Naphthalen-1-yl-ethylamino)-propyl]-N—((R)-2-oxo-tetrahydro-furan-3-yl)-benzamide (compound 1017). General procedure C was followed using (R)-4-[3-(1-naphthalen-1-yl-ethylamino)-propyl]-benzoic acid (compound 1010) and (R)-3-amino-dihydro-furan-2-one hydrochloride. The product was purified by HPLC-MS to afford the title compound. ¹³C NMR (75 MHz, DMSO) δ 175.72, 166.24, 164.12, 146.20, 139.96, 133.85, 131.38, 131.13, 129.11, 128.69, 128.62, 127.67, 126.42, 125.99, 125.90, 123.47, 123.33, 55.26, 53.31, 48.74, 46.54, 32.85, 30.42, 28.35, 23.06.

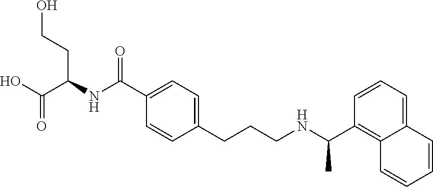

### Example 18

4-Hydroxy-2R-{4-[3-((R)-1-naphthalen-1-yl-ethylamino)-propyl]-benzoylamino}-butyric acid (compound 1018).

4-[3-((R)-1-Naphthalen-1-yl-ethylamino)-propyl]-N—((R)-2-oxo-tetrahydro-furan-3-yl)-benzamide (compound 1017) was hydrolysed with an excess of LiOH (5 eq.) in methanol-water (3:1). After stirring the mixture for 5.5 h at r.t., methanol was removed in vacuo, and residue was purified by flash chromatography in a gradient of methanol and DCM (0-80%) to afford the title compound. ¹³C NMR (75 MHz, DMSO) δ 174.08, 165.96, 145.25, 139.47, 133.38, 131.67, 130.67, 128.63, 128.20, 127.99, 127.20, 125.94, 125.52, 125.42, 123.01, 122.85, 57.82, 52.81, 50.45, 46.07, 34.03, 32.38, 29.99, 22.58.

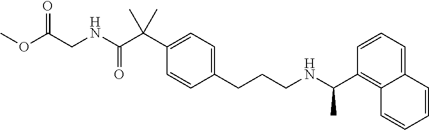

### Example 19

(R)-Methyl (2,2-dimethyl-2-{4-[3-(1-naphthalen-1-yl-ethylamino)-propyl]-phenyl}-acetoylamino)-acetate hydrochloride (compound 1019).

General procedure C was followed using (R)-2′,2′-dimethyl-2′-{4-[3-(1-naphthalen-1-yl-ethylamino)-propyl]-phenyl}-acetic acid hydrochloride (compound 1015) and amino-acetic acid methyl ester. The product was purified by flash chromatography in a gradient of methanol in DCM (0-10%) to afford the title compound. ¹³C NMR (151 MHz, DMSO) δ 176.40, 170.27, 143.03, 141.76, 140.01, 133.41, 130.84, 128.55, 127.86, 126.46, 125.64, 125.56, 125.14, 122.99, 122.63, 53.28, 51.40, 46.92, 45.55, 41.02, 32.46, 31.53, 26.86, 23.85.

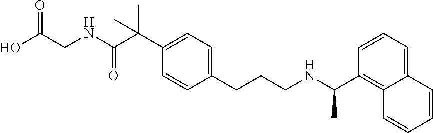

### Example 20

(R)-(2,2-Dimethyl-2-{4-[3-(1-naphthalen-1-yl-ethylamino)-propyl]-phenyl}-acetoylamino)-acetic acid (compound 1020).

Methyl(R)-(2,2-dimethyl-2-{4-[3-(1-naphthalen-1-yl-ethylamino)-propyl]-phenyl}-acetoylamino)-acetate hydrochloride (compound 1019) was hydrolyzed with an excess of lithium hydroxide (5 eq.) in methanol and water (3:1). After stirring for 3 h at rt, methanol was removed in vacuo, and dilute aqueous HCl was added to pH 2. The precipitate thus formed was filtered off and dried to afford the title compound. ¹³C NMR (151 MHz, DMSO) δ 175.41, 170.90, 143.21, 140.53, 139.71, 133.38, 130.75, 128.58, 127.91, 126.82, 125.77, 125.70, 125.55, 125.28, 122.93, 122.87, 52.98, 46.51, 45.51, 42.55, 32.27, 30.77, 26.87, 23.19.

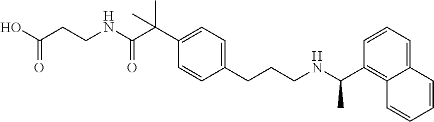

### Example 21

(R)-3-(2,2-Dimethyl-2-{4-[3-(1-naphthalen-1-yl-ethylamino)-propyl]-phenyl}-acetoylamino)-propionic acid (compound 1021). General procedure C was followed using (R)-2′,2′-dimethyl-2′-{4-[3-(1-naphthalen-1-yl-ethylamino)-propyl]-phenyl}-acetic acid, hydrochloride (compound 1015) as the acid and 3-amino-propionic acid methyl ester hydrochloride as the amine. The intermediate amide ester was purified by flash chromatography (DCM-methanol 9:1) to afford (R)-3-(2-methyl-2-{4-[3-(1-naphthalen-1-yl-ethylamino)-propyl]-phenyl}-propionylamino)-propionic acid methyl ester. The ester was hydrolysed with an excess of LiOH (5 eq.) in methanol-water (3:1). After stirring for 3 h at rt, methanol was removed in vacuo, and dilute aqueous HCl was added to pH 2. The precipitate thus formed was filtered off and dried to afford the title compound. ¹³C NMR (151 MHz, DMSO) δ 175.76, 173.15, 143.44, 139.25, 133.34, 130.56, 128.67, 127.96, 127.46, 126.14, 125.55, 123.26, 122.75, 52.49, 45.60, 35.18, 33.78, 31.90, 30.59, 29.59, 26.73, 26.65, 22.10.

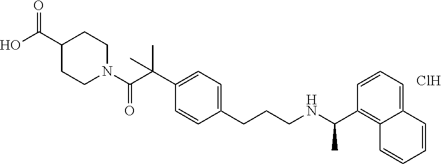

### Example 22

(R)-1-(2,2-Dimethyl-2-{4-[3-(1-naphthalen-1-yl-ethylamino)-propyl]-phenyl}-acetoyl)-piperidine-4-carboxylic acid hydrochloride (compound 1022). General procedure B was followed using (R)-2′,2′-dimethyl-2′-{4-[3-(1-naphthalen-1-yl-ethylamino)-propyl]-phenyl}-acetic acid, hydrochloride (compound 1015) as the acid and methyl 4-piperidinecarboxylate hydrochloride as the amine. The intermediate amide ester was purified by flash chromatography (DCM-methanol 9:1) to afford (R)-1-(2-methyl-2-{4-[3-(1-naphthalen-1-yl-ethylamino)-propyl]-phenyl}-propionyl)-piperidine-4-carboxylic acid methyl ester. The ester was hydrolysed with an excess of LiOH (5 eq.) in methanol-water (3:1). After stirring for 3 h at rt, methanol was removed in vacuo, and dilute aqueous HCl was added to pH 2. The precipitate thus formed was filtered off and dried to afford the title compound. ¹³C NMR (151 MHz, DMSO) δ 175.39, 173.21, 143.68, 138.45, 133.26, 130.29, 128.76, 128.45, 126.63, 125.92, 125.46, 124.43, 124.00, 122.58, 52.09, 48.47, 45.94, 44.93, 31.38, plus some very broad peaks between 28.5 and 27.0, and around 20.5.

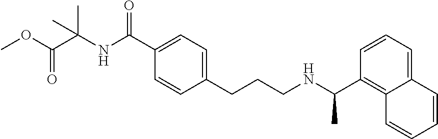

### Example 23

(R)-2-Methyl-2-{4-[3-(1-naphthalen-1-yl-ethylamino)-propyl]-benzoylamino}-propionic acid methyl ester (compound 1023). General procedure C was followed using (R)-4-[3-(1-naphthalen-1-yl-ethylamino)-propyl]-benzoic acid (compound 1010) as the acid and 2-amino-2-methyl-propionic acid methyl ester hydrochloride as the amine. The product was purified by flash chromatography (5-10% methanol in DCM) to afford the title compound. ¹³C NMR (151 MHz, DMSO) δ 174.40, 165.71, 145.90, 141.71, 133.41, 131.19, 130.83, 128.55, 127.91, 127.41, 126.49, 125.58, 125.56, 125.15, 122.99, 122.62, 55.32, 53.32, 51.69, 46.68, 32.70, 31.31, 24.88, 23.83.

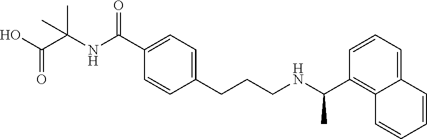

### Example 24

(R)-2-Methyl-2-{4-[3-(1-naphthalen-1-yl-ethylamino)-propyl]-benzoylamino}-propionic acid (compound 1024). (R)-2-Methyl-2-{4-[3-(1-naphthalen-1-yl-ethylamino}-propyl]-benzoylamino)-propionic acid methyl ester (compound 1023) was hydrolysed with an excess of LiOH (5 eq.) in methanol-water (3:1). After stirring for 3 h at rt, methanol was removed in vacuo, and dilute aqueous HCl was added to pH 2. The precipitate thus formed was filtered off and dried to afford the title compound. ¹³C NMR (151 MHz, DMSO) δ 175.76, 165.32, 144.48, 136.33, 133.29, 132.08, 130.38, 128.74, 128.09, 127.86, 127.33, 126.45, 125.78, 125.49, 123.79, 122.65, 55.40, 52.33, 45.26, 31.96, 28.16, 24.83, 21.04.

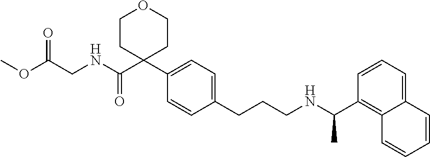

### Example 25

(R)-[(4-{4-[3-(1-Naphthalen-1-yl-ethylamino)-propyl]-phenyl}-tetrahydro-pyran-4-carbonyl)-amino]-acetic acid methyl ester (compound 1025). General procedure C was followed using (R)-4-(4-[3-(1-naphthalen-1-yl-ethylamino)-propyl]-phenyl)-tetrahydropyrane-4-carboxylic acid (compound 1016) as the acid and amino-acetic acid methyl ester hydrochloride as the amine. The product was purified by flash chromatography in a gradient of heptane and ethyl acetate to afford the title compound. ¹³C NMR (75 MHz, DMSO) δ 173.66, 141.75, 141.26, 140.42, 133.42, 130.85, 128.54, 128.06, 126.46, 125.55, 125.37, 125.13, 122.99, 122.64, 64.37, 53.27, 51.41, 47.36, 46.87, 40.97, 34.29, 32.40, 31.43, 23.81.

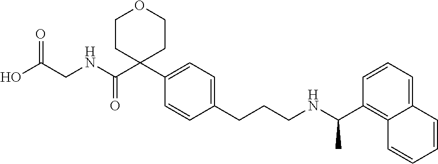

### Example 26

(R)-[(4-{4-[3-(1-Naphthalen-1-yl-ethylamino)-propyl]-phenyl}-tetrahydro-pyran-4-carbonyl)-amino]-acetic acid (compound 1026).

(R)-[(4-{4-[3-(1-Naphthalen-1-yl-ethylamino)-propyl]-phenyl}-tetrahydro-pyran-4-carbonyl)-amino]-acetic acid methyl ester (compound 1025) was hydrolysed with an excess of LiOH (5 eq.) in methanol-water (3:1). After refluxing the mixture for 1.5 h at 100° C., methanol was removed in vacuo, and dilute aqueous HCl was added to pH 5-6. The precipitate thus formed was filtered off and dried to afford the title compound as a white solid. ¹³C NMR (75 MHz, DMSO) δ 173.27, 171.26, 141.63, 139.55, 137.95, 133.34, 130.53, 128.68, 128.04, 127.63, 126.19, 125.60, 125.50, 123.35, 122.77, 64.34, 52.53, 47.36, 45.83, 41.32, 34.31, 31.86, 29.22, 21.81.

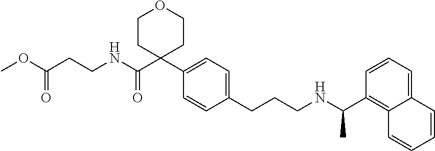

### Example 27

(R)-3-[(4-{4-[3-(1-Naphthalen-1-yl-ethylamino)-propyl]-phenyl}-tetrahydro-pyran-4-carbonyl)-amino]-propionic acid methyl ester (compound 1027). General procedure C was followed using (R)-4-(4-[3-(1-naphthalen-1-yl-ethylamino)-propyl]-phenyl)-tetrahydropyrane-4-carboxylic acid (compound 1016) as the acid and 3-amino-propionic acid methyl ester hydrochloride as the amine. The product was purified by flash chromatography in a gradient of DCM and ethyl acetate to afford the title compound. ¹³C NMR (75 MHz, DMSO) δ 173.01, 171.61, 141.74, 141.43, 140.30, 133.41, 130.85, 128.54, 128.07, 126.46, 125.55, 125.29, 125.13, 123.00, 122.64, 64.45, 53.27, 51.15, 47.37, 46.85, 35.27, 34.10, 33.36, 32.38, 31.39, 23.81.

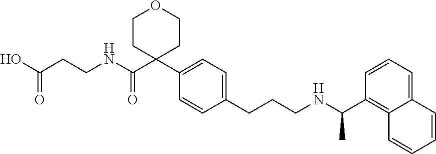

### Example 28

(R)-3-[(4-{4-[3-(1-Naphthalen-1-yl-ethylamino)-propyl]-phenyl}-tetrahydro-pyran-4-carbonyl)-amino]-propionic acid (compound 1028).

(R)-3-[(4-{4-[3-(1-Naphthalen-1-yl-ethylamino)-propyl]-phenyl}-tetrahydro-pyran-4-carbonyl)-amino]-propionic acid methyl ester (compound 1027) was hydrolysed with an excess of LiOH (5 eq.) in methanol-water (3:1). After refluxing the mixture for 1 h at 100° C., methanol was removed in vacuo, and dilute aqueous HCl was added to pH 5-6. The precipitate thus formed was filtered off and dried to afford the title compound as a white solid. ¹³C NMR (75 MHz, DMSO) δ 172.97, 141.62, 139.56, 133.34, 130.56, 128.67, 128.11, 127.53, 126.16, 125.56, 125.50, 125.44, 123.38, 122.75, 64.47, 52.51, 47.40, 45.65, 35.33, 34.10, 34.05, 33.73, 31.86, 29.31, 21.96.

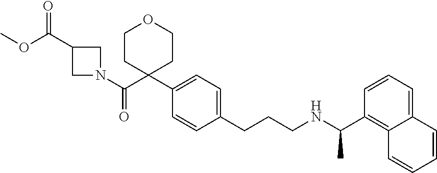

### Example 29

(R)-1-(4-{4-[3-(1-Naphthalen-1-yl-ethylamino)-propyl]-phenyl}-tetrahydro-pyran-4-carbonyl)-azetidine-3-carboxylic acid methyl ester (compound 1029). General procedure C was followed using (R)-4-(4-[3-(1-naphthalen-1-yl-ethylamino)-propyl]-phenyl)-tetrahydropyrane-4-carboxylic acid (compound 1016) as the acid and azetidine-3-carboxylic acid methyl ester hydrochloride as the amine. The product was purified by flash chromatography in a gradient of heptane and ethyl acetate to afford the title compound. ¹H NMR (300 MHz, DMSO) δ 8.31-8.22 (m, 1H), 7.94-7.87 (m, 1H), 7.76 (d, 1H), 7.68 (d, 1H), 7.55-7.43 (m, 3H), 7.19-7.09 (m, 4H), 4.52 (q, 1H), 4.04-3.91 (m, 1H), 3.89-3.77 (m, 1H), 3.75-3.62 (m, 3H), 3.61-3.44 (m, 6H), 3.28-3.15 (m, 2H), 2.67-2.52 (m, 2H), 2.50-2.31 (m, 2H), 2.21-2.07 (m, 2H), 1.89-1.65 (m, 4H), 1.36 (d, 3H).

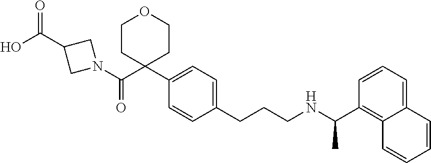

### Example 30

(R)-1-(4-{4-[3-(1-Naphthalen-1-yl-ethylamino)-propyl]-phenyl}-tetrahydro-pyran-4-carbonyl)-azetidine-3-carboxylic acid (compound 1030).

(R)-1-(4-{4-[3-(1-Naphthalen-1-yl-ethylamino)-propyl]-phenyl}-tetrahydro-pyran-4-carbonyl)-azetidine-3-carboxylic acid methyl ester (compound 1029) was hydrolysed with an excess of LiOH (5 eq.) in methanol-water (3:1). After refluxing the mixture for 3 h at 100° C., methanol was removed in vacuo, and dilute aqueous HCl was added to pH 5-6. The precipitate thus formed was filtered off and dried to afford the title compound as a white solid. ¹H NMR (500 MHz, DMSO) δ 8.25 (d, 1H), 7.89 (d, 1H), 7.78 (d, 1H), 7.70 (d, 1H), 7.54-7.42 (m, 3H), 7.13 (q, 4H), 4.73 (q, 1H), 3.82 (t, 2H), 3.77-3.53 (m, 6H), 3.17-3.06 (m, 1H), 2.70-2.52 (m, 4H), 2.17 (d, 2H), 1.90-1.75 (m, 4H), 1.49 (d, 3H).

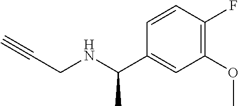

### Preparation 13

(R)—N-[1-(4-fluoro-3-methoxy-phenyl)ethyl]-prop-2-yn-1-amine. To a solution of (R)-1-(4-fluoro-3-methoxy-phenyl)-ethylamine (44 mmol) in DMSO (100 ml) was added Cs₂CO₃ followed by dropwise addition of propargyl bromide with vigorous stirring. After complete addition, the reaction mixture was stirred for 2 hours at rt and filtered. Brine, water and diethyl ether were added. The aqueous phase was separated and extracted three times with diethyl ether.

The combined organic extracts were dried over MgSO₄ and concentrated under reduced pressure to afford a yellow-brown oil. Chromatography afforded the title compound as a light yellow oil. ¹H NMR (300 MHz, CDCl3) δ 7.06-6.95 (m, 2H), 6.89-6.79 (m, 1H), 3.99 (q, J=6.5 Hz, 1H), 3.90 (s, 3H), 3.37 (dd, J=17.2, 1.2 Hz, 1H), 3.15 (d, J=17.1 Hz, 1H), 2.26-2.18 (m, 1H), 1.35 (d, J=6.5 Hz, 3H).

General procedure D (Sonogashira).

Aryl iodide (25 μmol, 1 eq.) and (R)—N-[1-(4-fluoro-3-methoxy-phenyl)ethyl]-prop-2-yn-1-amine (preparation 13) (28 μmol, 1.1 eq.) were dissolved in 0.5 ml diethylamine in a 4 ml vial. CuI (0.1 eq.) and Pd(PPh₃)₂Cl₂ (0.01 eq.) were added. The vial was flushed with argon, sealed with a screw cap and shaken overnight at r.t. The reaction mixture concentrated under reduced pressure and purified by reverse phase HPLC.

### General Procedure E (Catalytic Transfer Hydrogenation)

A solution of alkyne (0.10 mmol) and triethylsilane (0.80 mmol) in methanol (2 ml) containing Pd/C (10 mg) was stirred under argon. The hydrogenation was monitored by LC-MS and had generally reached full conversion after 1.5 hours. The catalyst was filtered off through a micropore filter, and the filtrate was concentrated under reduced pressure to afford the product after reverse phase preparative HPLC.

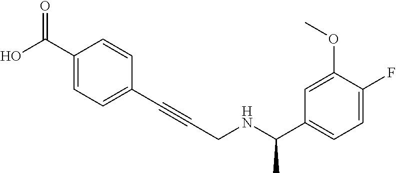

### Preparation 14

(R)-4-{3-[1-(4-Fluoro-3-methoxy-phenyl)-ethylamino]-prop-1-ynyl}-benzoic acid.

General procedure D was followed using 4-iodobenzoic acid as the aryl iodide.

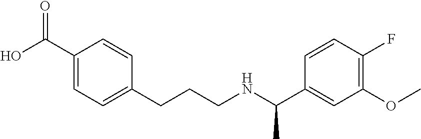

### Example 31

(R)-4-[3-[[1-(4-fluoro-3-methoxy-phenyl)ethyl]amino]-propyl]benzoic acid (compound 1031)

General procedure E was followed using (R)-4-{3-[1-(4-fluoro-3-methoxy-phenyl)-ethylamino]-prop-1-ynyl}-benzoic acid (Preparation 14) as the starting material. ¹H NMR (600 MHz, DMSO) δ 7.81 (d, 2H), 7.26 (d, 2H), 7.13 (dd, 1H), 7.09 (dd, 1H), 6.87-6.84 (m, 1H), 3.81 (s, 3H), 3.69-3.64 (m, 1H), 2.71-2.57 (m, 2H), 2.40-2.34 (m, 1H), 2.31-2.25 (m, 1H), 1.72-1.66 (m, 2H), 1.23 (d, 3H).

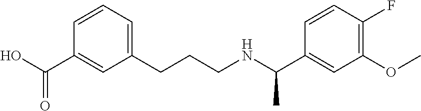

### Example 32

(R)-3-{3-[1-(4-Fluoro-3-methoxy-phenyl)-ethylamino]-propyl}-benzoic acid (compound 1032). The title compound was produced by successively carrying out the Sonogashira reaction following general procedure D with 3-iodobenzoic acid as the aryl iodide, and hydrogenation by following general procedure E. ¹H NMR (600 MHz, DMSO) δ 7.76 (br s, 1H), 7.72 (dt, 1H), 7.36-7.31 (m, 2H), 7.20 (dd, 1H), 7.11 (dd, 1H), 6.92-6.88 (m, 1H), 3.81 (s, 3H), 3.77 (q, 1H), 2.70-2.57 (m, 2H), 2.43 (dt, 1H), 2.34 (dt, 1H), 1.77-1.69 (m, 2H), 1.29 (d, 3H).

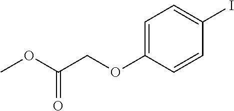

### Preparation 15

(4-Iodo-phenoxy)-acetic acid methyl ester.

5-Iodophenol (1.39 g, 6.34 mmol) was dissolved in DMF (6 ml) and treated with K₂CO₃ (1.31 g, 9.50 mmol) and methyl bromoacetate (1.16 g, 7.60 mmol) at room temperature over night. The reaction mixture was distributed between EtOAc and water. The aqueous phase was extracted with EtOAC and the combine organic phases were washed with brine. After drying and removal of solvents in vacuo, 1.84 g of the title compound was obtained. ¹H NMR (300 MHz, CDCl3) δ 7.61-7.53 (m, 2H), 6.71-6.65 (m, 2H), 4.61 (s, 2H), 3.80 (s, 3H).

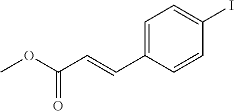

### Preparation 16

3-(4-Iodo-phenyl)-acrylic acid methyl ester.

(Triphenylphosphoranyliden)acetic acid methyl ester (1.44 g, 4.31 mmol) was dissolved in dry THF (5 ml) and treated with 4-iodobenzaldehyde (1 g, 4.31 mmol) at 50° C. over night. The reaction mixture was cooled, and petroleum ether was added. A white solid precipitated, which was filtered off. The filtrate was concentrated in vacuo, redissolved in EtOAc/petr. ether, and washed with water and brine. After drying and removal of solvents in vacuo, the residue was purified by chromatography (10% EtOAc/petr. ether to afford 1.13 g of the title compound as a white solid. ¹H NMR (300 MHz, CDCl3) δ 7.76-7.68 (m, 2H), 7.60 (d, J=16.0 Hz, 1H), 7.29-7.20 (m, 2H), 6.44 (d, J=16.0 Hz, 1H), 3.80 (s, 3H).

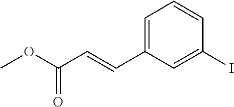

### Preparation 17

3-(3-Iodo-phenyl)-acrylic acid methyl ester.

(Triphenylphosphoranyliden)acetic acid methyl ester (1.44 g, 4.31 mmol) was dissolved in dry THF (5 ml) and treated with 3-iodobenzaldehyde (1 g, 4.31 mmol) at 50° C. over night. The reaction mixture was cooled, and petroleum ether was added. A white solid precipitated, which was filtered off. The filtrate was concentrated in vacuo, redissolved in EtOAc/petr. ether, and washed with water and brine. After drying and removal of solvents in vacuo, the residue was purified by chromatography (10% EtOAc/petr. ether to afford 0.87 g of the title compound. ¹H NMR (300 MHz, CDCl3) δ 7.87 (t, J=1.6 Hz, 1H), 7.74-7.68 (m, 1H), 7.58 (d, J=16.1 Hz, 1H), 7.47 (d, J=7.7 Hz, 1H), 7.12 (t, J=7.8 Hz, 1H), 6.41 (d, J=16.1 Hz, 1H), 3.81 (s, 3H).

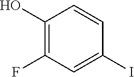

### Preparation 18

2-Fluoro-4-iodophenol.

2-Fluorophenol (1.12 g) was diluted in methanol, 15 ml, and sodium hydroxide (720 mg, 1.8 eq.) was added. When the alkali had dissolved, the reaction mixture was cooled to 0° C. and iodine (2.54 g, 1 eq.) was added. After 5 min. the mixture was worked up with EtOAc and water. The organics were dried, concentrated in vacuo and purified by chromatography (7% EtOAc in Heptane). 714 mg obtained. P ¹H NMR (300 MHz, CDCl3) δ 7.39 (m, 1H), 7.33 (m, 1H), 6.76 (m, 1H), 5.23 (br, 1H).

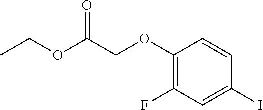

### Preparation 19

(2-Fluoro-4-iodo-phenoxy)-acetic acid ethyl ester.

2-Fluoro-4-iodophenol (Preparation 18, 628 mg) was dissolved in DMF (5 ml) and treated with ethyl bromoacetate (2730, 1.05 eq.) and potassium carbonate (470 mg, 1,2 eq.) over night at room temperature. After distribution between EtOAc and water, the organics were dried and concentrated in vacuo down to 0.02 mmHg. 770 mg crystalline material was obtained. ¹H NMR (300 MHz, CDCl3) δ 7.26 (m, 1H), 7.17 (m, 1H), 6.82 (t, 1H), 4.66 (s, 2H), 4.26 (t, 2H), 1.29 (t, 3H).

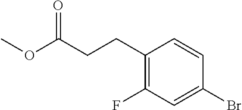

### Preparation 20

Methyl 2-Fluoro-4-bromophenylpropanoate.

2-Fluoro-4-bromophenylpropanoic acid (1 g, 4.05 mmol) was dissolved in DMF (8 ml) and treated with methyl iodide (350 1.4 eq.) and potassium carbonate (1 g, 1.8 eq.) over night at room temperature. The reaction mixture was distributed between EtOAc and water. The aqueous phase was extracted with EtOAc, and the organic phases were combined and washed with water. After drying and removal of solvents in vacuo, 1.03 g pure product was obtained. ¹H NMR (300 MHz, CDCl3) 7.38 (m, 2H), 6.95 dd, 1H), 3.66 (s, 3H), 2.92 (t, 2H), 2.61 (t, 2H).

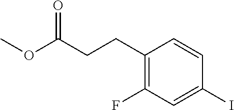

### Preparation 21

Methyl 2-fluoro-4-iodophenylpropanoate

Methyl 2-fluoro-4-bromophenylpropanoate (Preparation 20) 1.03 g, 3.95 mmol), copper iodide (38 mg, 0.05 eq.) and sodium iodide (1.2 g, 2 eq.) was placed in a dry 25 round bottom flask under argon. N,N′-dimethyl-ethylenediamine (43 μl, 0.1 eq.) and dry dioxane (5 ml) were added and heated at 110° C. over night. After aqueous work up with EtOAc, a crude product was obtained, containing 9% residual bromo-substrate. The iodoaromatic was used as such. ¹H NMR (300 MHz, CDCl3) δ 7.43-7.34 (m, 2H), 6.95 (t, 1H), 3.66 (s, 3H), 2.92 (t, J=7.6 Hz, 2H), 2.61 (t, J=7.6 Hz, 2H).

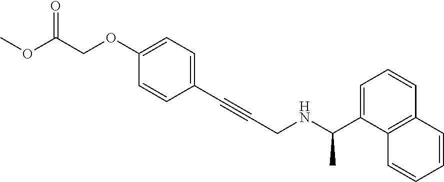

### Preparation 22

(R)-{4-[3-(1-Naphthalen-1-yl-ethylamino)-prop-1-ynyl]-phenoxy}-acetic acid methyl ester.

General procedure A was followed using (4-iodo-phenoxy)-acetic acid methyl ester as the aryl iodide.

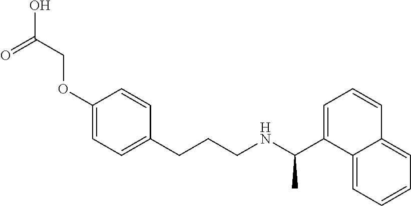

### Example 33

(R)-{4-[3-(1-Naphthalen-1-yl-ethylamino)-propyl]-phenoxy}-acetic acid (compound 1033)

General procedure E was followed using (R)-{4-[3-(1-naphthalen-1-yl-ethylamino)-prop-1-ynyl]-phenoxy}-acetic acid methyl ester (preparation 22) as the alkyne. The intermediate methyl ester was hydrolyzed with 20% NaOH in methanol (over night at room temperature) and purified by HPLC to afford the title compound. ¹H NMR (600 MHz, DMSO) δ 8.24 (d, 1H), 7.95-7.92 (m, 1H), 7.81 (d, 1H), 7.73 (d, 1H), 7.56-7.48 (m, 3H), 7.00 (d, 2H), 6.72 (d, 2H), 4.73-4.66 (m, 1H), 4.41 (s, 2H), 2.59-2.41 (m, 4H), 1.71 (p, 2H), 1.42 (d, 3H).

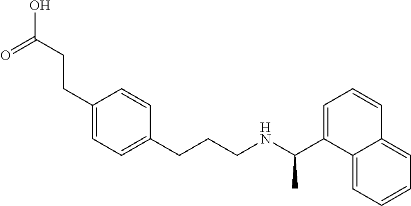

### Example 34

(R)-3-{4-[3-(1-Naphthalen-1-yl-ethylamino)-propyl]-phenyl}-propionic acid (compound 1034). The title compound was produced by carrying out the Sonogashira reaction following general procedure A using 3-(4-iodo-phenyl)-acrylic acid methyl ester as the aryl iodide, and then successively carrying out the reactions of hydrogenation and hydrolysis in the same manner as in example 33. ¹H NMR (600 MHz, DMSO) δ 8.26 (d, 1H), 7.94-7.89 (m, 1H), 7.77 (d, 1H), 7.69 (d, 1H), 7.54-7.45 (m, 3H), 7.07 (d, 2H), 7.03 (d, 2H), 4.54 (q, 1H), 2.75 (t, 2H), 2.61-2.34 (m, 6H), 1.75-1.64 (m, 2H), 1.36 (d, 3H).

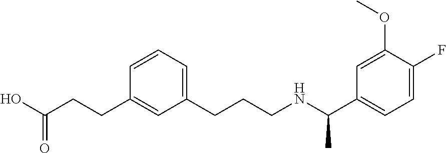

### Example 35

(R)-3-(3-{3-[1-(4-Fluoro-3-methoxy-phenyl)-ethylamino]-propyl}-phenyl)-propionic acid (compound 1035)

The title compound was produced by carrying out the Sonogashira reaction following general procedure D using 3-(3-iodo-phenyl)-acrylic acid methyl ester (preparation 17) as the aryl iodide, and then successively carrying out the reactions of hydrogenation and hydrolysis in the same manner as in example 33. ¹H NMR (600 MHz, DMSO) δ 7.15-7.12 (m, 2H), 7.09 (dd, 1H), 7.01-6.94 (m, 3H), 6.86 (ddd, 1H), 3.81 (s, 3H), 3.66 (q, 1H), 2.75 (t, 2H), 2.59-2.45 (m, 2H), 2.46 (t, 2H), 2.36 (dt, 1H), 2.28 (dt, 1H), 1.65 (p, 2H), 1.22 (d, 3H).

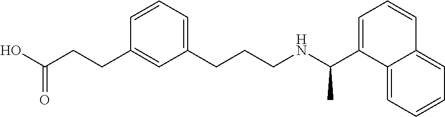

### Example 36

(R)-3-{3-[3-(1-Naphthalen-1-yl-ethylamino)-propyl]-phenyl}-propionic acid (compound 1036)

The title compound was produced by carrying out the Sonogashira reaction following general procedure A using 3-(3-iodo-phenyl)-acrylic acid methyl ester (preparation 17) as the aryl iodide, and then successively carrying out the reactions of hydrogenation and hydrolysis in the same manner as in example 33. ¹H NMR (600 MHz, DMSO) δ 8.27 (d, 1H), 7.94-7.89 (m, 1H), 7.77 (d, 1H), 7.69 (d, 1H), 7.55-7.46 (m, 3H), 7.15-7.09 (m, 1H), 7.01-6.92 (m, 3H), 4.54 (q, 1H), 2.74 (t, 2H), 2.62-2.34 (m, 6H), 1.76-1.54 (m, 2H), 1.37 (d, 3H).

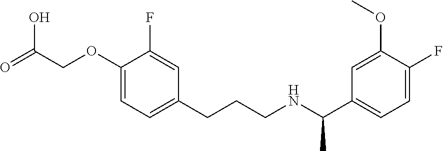

### Example 37

(R)-2-[2-fluoro-4-[3-[[1-(4-fluoro-3-methoxy-phenyl)ethyl]amino]propyl]phenoxy]acetic acid (compound 1037)

The title compound was produced by carrying out the Sonogashira reaction following general procedure D using (2-fluoro-4-iodo-phenoxy)-acetic acid ethyl ester (Preparation 19) as the aryl iodide, and then successively carrying out the reactions of hydrogenation and hydrolysis in the same manner as in example 33. ¹H NMR (600 MHz, DMSO) δ 7.36 (d, J=7.4 Hz, 1H), 7.18 (dd, J=11.4, 8.3 Hz, 1H), 7.01-6.97 (m, 1H), 6.95 (dd, J=12.6, 1.9 Hz, 1H), 6.80 (t, J=8.7 Hz, 1H), 6.71 (d, J=8.5 Hz, 1H), 4.42 (s, 2H), 4.06 (q, 1H), 2.66-2.57 (m, 1H), 2.54 (s, 3H), 2.50-2.38 (m, 3H), 1.79-1.68 (m, 2H), 1.44 (d, J=6.7 Hz, 3H).

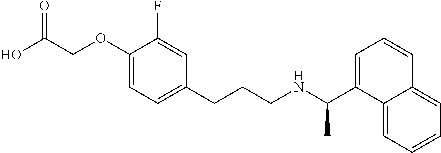

### Example 38

(R)-{2-Fluoro-4-[3-(1-naphthalen-1-yl-ethylamino)-propyl]-phenoxy}-acetic acid (compound 1038)

The title compound was produced by carrying out the Sonogashira reaction following general procedure A using (2-fluoro-4-iodo-phenoxy)-acetic acid ethyl ester (Preparation 19) as the aryl iodide, and then successively carrying out the reactions of hydrogenation and hydrolysis in the same manner as in example 33. ¹H NMR (600 MHz, DMSO) δ 8.23 (d, J=8.4 Hz, 1H), 7.98-7.94 (m, 1H), 7.87 (d, J=8.2 Hz, 1H), 7.79 (d, J=7.1 Hz, 1H), 7.59-7.51 (m, 3H), 6.96 (dd, J=12.6, 1.9 Hz, 1H), 6.83 (t, J=8.7 Hz, 1H), 6.76 (d, J=8.3 Hz, 1H), 4.96-4.87 (m, 1H), 4.48 (s, 2H), 2.74-2.65 (m, 1H), 2.57-2.42 (m, 3H), 1.80-1.72 (m, 2H), 1.51 (d, J=6.6 Hz, 3H).

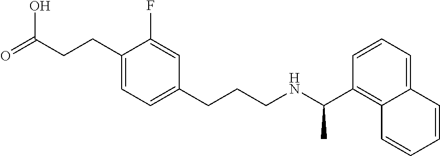

### Example 39

(R)-3-{2-Fluoro-4-[3-(1-naphthalen-1-yl-ethylamino)-propyl]-phenyl}-propionic acid (compound 1039)

The title compound was produced by carrying out the Sonogashira reaction following general procedure A using methyl 2-fluoro-4-iodophenylpropanoate (Preparation 21) as the aryl iodide, and then successively carrying out the reactions of hydrogenation and hydrolysis in the same manner as in example 33.1H NMR (600 MHz, DMSO) δ 8.26 (d, J=8.2 Hz, 1H), 7.94-7.90 (m, 1H), 7.78 (d, J=8.1 Hz, 1H), 7.69 (d, J=7.0 Hz, 1H), 7.54-7.47 (m, 3H), 7.14 (t, J=8.0 Hz, 1H), 6.91 (dd, J=15.5, 9.6 Hz, 2H), 4.57 (q, J=6.4 Hz, 1H), 2.77 (t, J=7.7 Hz, 2H), 2.63-2.44 (m, 3H), 2.49-2.46 (m, 3H), 2.43-2.36 (m, 1H), 1.74-1.67 (m, 2H), 1.37 (d, J=6.6 Hz, 3H).

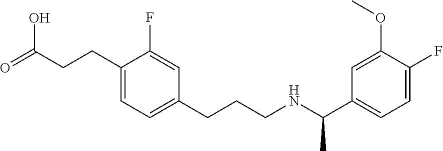

### Example 40

(R)-3-(2-Fluoro-4-{3-[1-(4-fluoro-3-methoxy-phenyl)-ethylamino]-propyl}-phenyl)-propionic acid (compound 1040)

The title compound was produced by carrying out the Sonogashira reaction following general procedure D using methyl 2-fluoro-4-iodophenylpropanoate (Preparation 21) as the aryl iodide, and then successively carrying out the reactions of hydrogenation and hydrolysis in the same manner as in example 33. 1H NMR (600 MHz, DMSO) δ 7.16 (t, J=8.1 Hz, 2H), 7.10 (dd, J=11.5, 8.2 Hz, 1H), 6.94-6.89 (m, 2H), 6.89-6.85 (m, 1H), 3.82 (s, 3H), 3.72 (dd, J=13.2, 6.6 Hz, 1H), 2.78 (t, J=7.7 Hz, 2H), 2.62-2.49 (m, 2H), 2.47 (t, J=7.7 Hz, 2H), 2.43-2.36 (m, 1H), 2.32-2.27 (m, 1H), 1.70-1.64 (m, 2H), 1.25 (d, J=6.6 Hz, 3H).

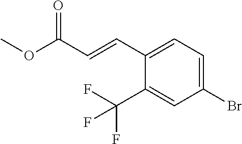

### Preparation 23

3-(4-Bromo-2-trifluoromethyl-phenyl)-acrylic acid methyl ester. A solution of methyl (triphenylphosphoranylidene) acetate (1.32 g, 3.95 mmol) and 4-bromo-2-trifluoromethylbenzaldehyde (1.0 g, 3.95 mmol) in THF (5 ml) was heated over night at 50° C. Petr. ether was added to the reaction mixture, and the resulting precipitate was filtered off. The filtrate was concentrated under reduced pressure to afford a crude mixture of the title compound (trans, 85%) and the corresponding cis isomer (15%).

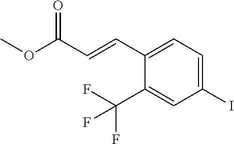

### Preparation 24

3-(4-Iodo-2-trifluoromethyl-phenyl)-acrylic acid methyl ester.

A mixture of 3-(4-bromo-2-trifluoromethyl-phenyl)-acrylic acid methyl ester (3.95 mmol), NaI (1.18 g, 7.9 mmol), CuI (38 mg, 0.20 mmol) and N,N′-dimethylethylenediamine (35 mg, 0.40 mmol) in 5 ml dioxane was heated at 110° C. over night. The reaction mixture was cooled and distributed between EtOAc and water/brine. The organic phase was dried, filtered and concentrated in vacuo to afford the title compound. (85% trans, 15% cis).

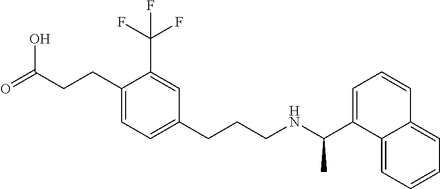

### Example 41

(R)-3-[4-[3-[[1-(1-naphthyl)ethyl]amino]propyl]-2-(trifluoromethyl)phenyl]propanoic acid (compound 1041)

The title compound was produced by carrying out the Sonogashira reaction following general procedure A using 3-(4-iodo-2-trifluoromethyl-phenyl)-acrylic acid methyl ester (preparation 24) as the aryl iodide, and then successively carrying out the reactions of hydrogenation and hydrolysis in the same manner as in example 33.

1. A compound of general formula I 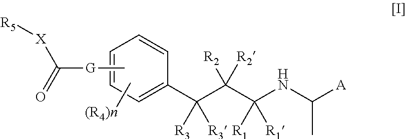
wherein
A is Ci-ioheteroaryl, C₆₋₁₄aryl or Ce-ioheterocycloalkylaryl, optionally substituted by one or more substituents independently at each occurrence selected from the group consisting of halogen, hydroxy, trifluoromethyl, amino, Ci-₆-alkyl, C₂-̂-alkenyl, Ci₋₆-alkyloxy, or Ci₋₆-alkenyloxy, wherein said Ci₋₆-alkyl, C₂-₆-alkenyl, Ci-₆-alkyloxy, or C₂₋₆-alkenyloxy may be optionally substituted by one or more substituents selected from halogen, hydroxy, trifluoromethyl and NH₂;
Ri_{/}Ri′i R2, R2′₁ R3 and R3′, independently of each other, are selected from the group consisting of hydrogen, halogen, hydroxy, trifluoromethyl, amino, Cₓ₋₆-alkyl, C₂-₆-alkenyl, Ci-₆-alkyloxy, and C₂-₆-alkenyloxy;
R₄, independently at each occurrence, is selected from the group consisting of hydrogen, halogen, hydroxy, trifluoromethyl, amino, Ci₋₆-alkyl, C₂₋₆-alkenyl, Ci₋₆-alkyloxy, and Ci₋₆-alkenyloxy, wherein said Ci₋₆-alkyl, C₂₋₆-alkenyl, Ci₋₆-alkyloxy, or C₂₋₆-alkenyloxy may be optionally substituted by one or more substituents selected from halogen, hydroxy, trifluoromethyl and NH₂;
n is an integer from 0 to 3;
R₅ is hydrogen or is selected from the group consisting of Ci-₆-alkyl, C₂₋₆-alkenyl, Ci₋₆-alkyloxy, and Ci-₆-alkenyloxy, Ci₋₅-heterocyclyl, Ci₋₅-heterocyclyl-Ci-₆-alkyl and Ci-₅-heterocyclyl-C₂₋₆-alkenyl, optionally substituted by one or more substituents independently at each occurrence selected from the group consisting of halogen, hydroxy, trifluoromethyl, amino, Ci₋₆-alkyl, C₂₋₆-alkenyl, Ci₋₆-alkyloxy, and Ci-₆-alkenyloxy, wherein said Ci₋₆-alkyl, C₂₋₆-alkenyl, Ci-₆-alkyloxy, or C₂₋₆-alkenyloxy may be optionally substituted by one or more substituents selected from halogen, hydroxy, trifluoromethyl and NH₂, or
R₅ is C₃-₄-alkylene and together with —C(O)—O forms a 5 or 6 membered cyclic or heterocyclic ring attached to G;
X is O;
G is a direct bond or is selected from the group consisting of Ci₋₆-alkylene, -0-Ci-₆-alkylene, C₂₋₆-alkenylene, —O—C₂₋₆-alkenylene, Ci-s-heterocyclylene, —R₆—CONH—R₈, or —R₇—CO—R₉;
R₆, R₇, R₈ and R₉, independently of each other, is a direct bond or is Ci₋₆-alkylene, C₂₋₆-alkenylene, C₃₋₆-cycloalkylene or Ci-s-heterocyclylene, wherein said Ci₋₆-alkylene, C₂₋₆-alkenylene, C₃₋₆-cycloalkylene or Ci-₅-heterocyclylene is optionally substituted by one or more substituents independently at each occurrence selected from the group consisting of halogen, hydroxy, trifluoromethyl, amino, Ci-₆-alkyl, C₂-6-alkenyl, Ci₋₆-alkyloxy, or Ci₋₆-alkenyloxy, wherein said Ci₋₆-alkyl, C₂-₆-alkenyl, Ci₋₆-alkyloxy, or C₂-₆-alkenyloxy may be optionally substituted by one or more substituents selected from halogen, hydroxy, trifluoromethyl and NH₂;
as well as stereoisomers, pharmaceutically acceptable salts, solvates, hydrates, or in vivo hydrolysable esters thereof.

2. A compound of general formula Ia 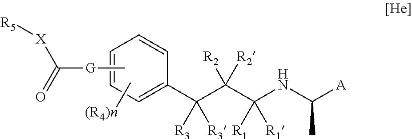
wherein
A is Ci-ioheteroaryl, C₆₋i₄aryl or Cβ-ioheterocycloalkylaryl, optionally substituted by one or more substituents independently at each occurrence selected from the group consisting of halogen, hydroxy, trifluoromethyl, amino, Ci-₆-alkyl, C₂₋₆-alkenyl, Ci-₆-alkyloxy, or Ci₋₆-alkenyloxy, wherein said Ci-₆-alkyl, C₂-₆-alkenyl, Ci-₆-alkyloxy, or C₂-₆-alkenyloxy may be optionally substituted by one or more substituents selected from halogen, hydroxy, trifluoromethyl and NH₂;
Ri, Ri′_{/} R_{2/} R_{2/} R₃ and R₃′, independently of each other, are selected from the group consisting of hydrogen, halogen, hydroxy, trifluoromethyl, amino, Ci₋₆-alkyl, C₂₋₆-alkenyl, Ci-₆-alkyloxy, and C₂-₆-alkenyloxy;
R₄, independently at each occurrence, is selected from the group consisting of hydrogen, halogen, hydroxy, trifluoromethyl, amino, Ci-₆-alkyl, C₂-₆-alkenyl, Ci₋₆-alkyloxy, and Ci-₆-alkenyloxy, wherein said Ci-₆-alkyl, C₂₋₆-alkenyl, Ci-₆-alkyloxy, or C₂-₆-alkenyloxy may be optionally substituted by one or more substituents selected from halogen, hydroxy, trifluoromethyl and NH₂;
n is an integer from 0 to 3;
R₅ is hydrogen or is selected from the group consisting of Ci₋₆-alkyl, C₂-₆-alkenyl, C₁₋₆-alkyloxy, and Ci-₆-alkenyloxy, Ci-₅-heterocyclyl, Ci-₅-heterocyclyl-Ci-₆-alkyl and C₁-₅-heterocyclyl-C₂-₆-alkenyl, optionally substituted, by one or more substituents independently at each occurrence selected from the group consisting of halogen, hydroxy, trifluoromethyl, amino, Ci-₆-alkyl, C₂₋₆-alkenyl, Ci₋₆-alkyloxy, and Ci-₆-alkenyloxy, wherein said Cχ-₆-alkyl, C₂₋₆-alkenyl, Ci-₆-alkyloxy, or C₂₋₆-alkenyloxy may be optionally substituted by one or more substituents selected from halogen, hydroxy, trifluoromethyl and NH₂, or
R₅ is C₃-₄-alkylene and together with the adjacent —C(O)—O forms a 5 or 6 membered cyclic or heterocyclic ring attached to G;
X is O;
G is a direct bond or is selected from the group consisting of Ci₋₆-alkylene, —O—C-₆-alkylene, C₂-₆-alkenylene, —O—C₂-₆-alkenylene, Ci₅-heterocyclylene, —R₆—CONH—R₈, or —R₇—CO—R₉;
R_{δ}, R₇, Re and R₉, independently of each other, is a direct bond or is Ci-₆-alkylene, C₂-₆-alkenylene, C₃-₆-cycloalkylene or Ci-₅-heterocyclylene, wherein said C₁₋₆-alkylene, C₂₋₆-alkenylene, C₃-₆-cycloalkylene or Ci-₅-heterocyclylene is optionally substituted by one or more substituents independently at each occurrence selected from the group consisting of halogen, hydroxy, trifluoromethyl, amino, Ci-₆-alkyl, C₂₋₆-alkenyl, Ci₋₆-alkyloxy, or Ci-₆-alkenyloxy, wherein said C₁₋₆-alkyl, C₂₋₆-alkenyl, Ci-₆-alkyloxy, or C₂₋₆-alkenyloxy may be optionally substituted by one or more substituents selected from halogen, hydroxy, trifluoromethyl and NH₂;
as well as stereoisomers, pharmaceutically acceptable salts, solvates, hydrates, or in vivo hydrolysable esters thereof.

3. The compound according to claim 1 or 2, wherein A is naphthyl.

4. The compound according to claim 1 or 2, wherein A is 4-fluoro-3-methoxy-phenyl.

5. The compound according claim 1, wherein R₁, R₁′, R₂, R₂′, R₃ and R₃′ are all hydrogen.

6. The compound according to claim 1, wherein at least one of R₁, R₁′, R2_{/} R2_{/} R3 and R₃′ is methyl.

7. The compound according to claim 1, wherein at least one R₁, R₁′, R₂, R₂, R₃ and R₃ is hydroxy.

8. The compound according to claim 1, wherein n is 0.

9. The compound according to claim 1, wherein n is 1.

10. The compound according to claim 9, wherein R₄ is hydroxy.

11. The compound according to claim 9, wherein R₄ is F.

12. The compound according to claim 9, wherein R₄ is trifluoromethyl.

13. The compound according to claim 1, wherein R₅ is hydrogen.

14. The compound according to claim 1, wherein R₅ is selected from the group consisting of methyl, ethyl, n-propyl, isopropyl, n-butyl, isobutyl, sec-butyl and tert-butyl.

15. The compound according to claim 1, wherein R₅ is morpholino-ethyl.

16. The compound according to claim 1, wherein R₅ together with the adjacent —C(O)—O— represents 2-oxo-tetrahydrofuranyl.

17. The compound according to claim 1, wherein G is a direct bond.

18. The compound according to claim 1, wherein G is ethylene or methoxy.

19. The compound according to claim 1, wherein G is isopropylene, O-isopropylene, or tetrahydropyranylene.

20. The compound according to claim 1, wherein G is —R₆—CONH—R₈-

21. The compound according to claim 20, wherein R₆ is a direct bond.

22. The compound according to claim 21, wherein R₆ is Ci-₆-alkylene or heterocyclylene.

23. The compound according to claim 22, wherein R₆ is isopropylene or tetrahydropyranylene.

24. The compound according to any of the claims 20-23, wherein R₈ is Ci-₆-alkylene.

25. The compound according to claim 24, wherein R₈ is methylene, ethylene, n-propylene or isopropylene, optionally substituted with one or more substituents selected from halogen, hydroxy, amino, trifluoromethyl, Ci-C₄-alkyl, Ci₋₄-alkoxy or hydroxy-Ci-C₄-alkyl.

26. The compound according to claim 1, wherein G is —R₇—CO—R₉.

27. The compound according to claim 26, wherein R₇ is Ci₋₆-alkylene or heterocyclylene.

28. The compound according to claim 27, wherein R₇ is isopropylene or tetrahydropyranylene.

29. The compound according to any of the claims 26-28, wherein R₉ is heterocyclylene.

30. The compound according to claim 29, wherein R₉ is piperidino or azetidino.

31. The compound according to claim 1 or 2 selected from the group comprising:
Ethyl(R)-3-(3-{1-naphthalen-1-yl-ethylamino}-propyl)-benzoate, hydrochloride (compound 1001), (ΛJ-Z-Hydroxy-S-tS-Cl-naphthalen-1-yl-ethylaminoJ-propyπ-benzoic acid methyl ester hydrochloride (compound 1002),
(RJ-Z-Hydroxy-S-tS-Cl-naphthalen-1-yl-ethylaminoJ-propylj-benzoic acid (compound 1003),
Methyl(R)-2-[3-(1-naphthalene-1-yl-ethylamino)-propyl]benzoate hydrochloride (compound 1004),
2-[3-((R)-1-naphthalene-1-yl-ethylamino)-propyl]-benzoic acid hydrochloride (compound 1005),
(R)-2-fluoro-5-[3-(1-naphthalene-1-yl-ethylamino)-propyl]-benzoic acid (compound 1006),
4-[3-((R)-1-naphthalene-1-yl-ethylamino)-butyl]-benzoic acid methyl ester (compound 1007), tert-Butyl(R)-(2,2-Dimethyl-2-{4-[3-(1-naphthalen-1-yl-ethylamino)-propyl]-phenoxy}-acetoylamino)-acetate (compound 1008),
(R)-(2,2-Dimethyl-2-{4-[3-(1-naphthalen-1-yl-ethylamino)-propyl]-phenoxy}-acetoylamino)-acetic acid, hydrochloride (compound 1009),
(R)-4-[3-(1-Naphthalen-1-yl-ethylamino)-propyl]-benzoic acid (compound 1010),
(R)-3-(3-{1-naphthalen-1-yl-ethylamino}-propyl)-benzoic acid (compound 1011),
(R)-4-[3-(1-Naphthalen-1-yl-ethylamino)-propyl]-benzoic acid 2-morpholin-4-yl-ethyl ester hydrochloride (compound 1012),
Ethyl 4-(2-hydroxy-3-{(R)-1-naphthalene-1-yl-ethylamino}-propyl)-benzoate (compound 1013),
4-(2-hydroxy-3-{(R)-1-naphthalen-1-yl-ethylamino}-propyl)-benzoic acid (compound 1014),
(R)-2′,2′-Dimethyl-2′-{4-[3-(1-naphthalen-1-yl-ethylamino)-propyl]-phenyl}-acetic acid, hydrochloride (compound 1015),
(R)-4-(4-[3-(1-naphthalen-1-yl-ethylamino)-propyl]-phenyl)-tetrahydropyrane-4-carboxylic acid (compound 1016),
4-[3-((R)-1-naphthalene-1-yl-ethylamino)-propyl]-N-((R)-2-oxo-tetrahydro-furan-3-yl)-benzamide (Compound 1017),
4-Hydroxy-2R-{4-[3-((R)-1-naphthalene-1-yl-ethylamino)-propyl]-benzoylamino}-butyric acid (Compound 1018),
(R)-Methyl (2,2-dimethyl-2-{4-[3-(1-naphthalen-1-yl-ethylamino)-propyl]-phenyl}-acetoylamino)-acetate hydrochloride (compound 1019),
(R)-(2,2-Dimethyl-2-{4-[3-(1-naphthalen-1-yl-ethylamino)-propyl]-phenyl}-acetoylamino)-acetic acid (compound 1020),
(R)-3-(2,2-Dimethyl-2-{4-[3-(1-naphthalen-1-yl-ethylamino)-propyl]-phenyl}-acetoylamino)-propionic acid (compound 1021), (R)-1-(2,2-Dimethyl-2-{4-[3-(1-naphthalen-1-yl-ethylamino)-propyl]-phenyl}-acetoyl)-piperidine-4-carboxylic acid; hydrochloride (compound 1022), (R)-2-Methyl-2-{4-[3-(1-naphthalen-1-yl-ethylamino)-propyl]benzoylamino}-propionic acid methyl ester (compound 1023),
(R)-2-Methyl-2-{4-[3-(1-naphthalen-1-yl-ethylamino)-propyl]-benzoylamino}-propionic acid (compound 1024),
(R)-[(4-{4-[3-(1-Naphthalen-1-yl-ethylamino)-propyl]-phenyl}-tetrahydro-pyran-4-carbonyl)-amino]-acetic acid methyl ester (compound 1025), (R)-[(4-{4-[3-(1-Naphthalen-1-yl-ethylamino)-propyl]-phenyl}-tetrahydro-pyran-4-carbonyl)-amino]-acetic acid (compound 1026),
acid methyl ester (Compound 1027), acid (Compound 1028), acid methyl ester (Compound 1029), acid (Compound 1030), (R)-4-[3-[[1-(4-fluoro-3-methoxy-phenyl)ethyl]amino]propyl]benzoic acid (Compound 1031),
(R)-3-{3-[1-(4-Fluoro-3-methoxy-phenyl)-ethylamino]-propyl}-benzoic acid (compound 1032),
(R)-{4-[3-((1-Naphthalen-1-yl-ethylamino)-propyl]-phenoxy}-acetic acid (compound 1033),
(R)-3-{4-[3-(1-Naphthalen-1-yl-ethylamino)-propyl]-phenyl}-propionic acid (compound 1034)
(R)-3-(3-{3-[1-(4-Fluoro-3-methoxy-phenyl)-ethylamino]-propyl}-phenyl)-propionic acid (compound 1035),
(R)-3-{3-[3-(1-Naphthalen-1-yl-ethylamino)-propyl]-phenyl}-propionic acid (compound 1036),
(R)-2-[2-fluoro-4-[3-[[1-(4-fluoro-3-methoxy-phenyl)ethyl]amino]-propyl]phenoxy]acetic acid (compound 1037),
(R)-{2-Fluoro-4-[3-(1-naphthalen-1-yl-ethylamino)-propyl]-phenoxy}-acetic acid (compound 1038),
(R)-3-{2-Fluoro-4-[3-(1-naphthalen-1-yl-ethylamino)-propyl]-phenyl}-propionic acid (compound 1039),
(R)-3-(2-Fluoro-4-{3-[1-(4-fluoro-3-methoxy-phenyl)-ethylamino]-propyl}-phenyl)-propionic acid (compound 1040), and (RJ-S-̂-tS-tCl-Cl-naphthyOethyπaminoJpropyπ-Z-Ctrifluoromethyl)-phenyljpropanoic acid (compound 1041).

32. The compound according to claim 1 for use as a medicament in therapy.

33. The compound according to claim 1 for use in the treatment, amelioration or prophylaxis of physiological disorders or diseases associated with disturbances of CaSR activity.

34. Use of a compound according to claim 1 for the manufacture of a medicament for the prophylaxis, treatment or amelioration of physiological disorders or diseases associated with disturbances of CaSR activity.

35. A pharmaceutical composition comprising a compound according to claim 1 or a pharmaceutically acceptable salt, solvate, -or in vivo hydrolysable ester thereof together with a pharmaceutically acceptable vehicle or excipient.

36. A method of preventing, treating or ameliorating parathyroid carcinoma, parathyroid adenoma, primary parathyroid hyperplasia, cardiac, renal or intestinal disfunctions, diseases of the central nervous system, chronic renal failure, chronic kidney disease, polycystic kidney disorder, podocyte-related diseases, primary hyperparathyroidism, secondary hyperparathyroidism, tertiary hyperparathyroidism, anemia, cardiovascular diseases, renal osteodystrophy, osteitis fibrosa, adynamic bone disease, osteoporosis, steroid induced osteoporosis, senile osteoporosis, post menopausal osteoporosis, osteomalacia and related bone disorders, bone loss post renal transplantation, cardiovascular diseases, gastrointestinal diseases, endocrine and neurodegenerative diseases, cancer, Alzheimer's disease, IBS, IBD, malassimilation, malnutrition, abnormal intestinal motility such as diarrhea, vascular calcification, abnormal calcium homeostasis, hypercalcemia, or renal bone diseases, the method comprising administering to a patient in need thereof an effective amount of a compound according to claim 1, optionally in combination or as supplement with an active vitamin-D sterol or vitamin-D derivative, such as 1-α-hydroxycholecalciferol, ergocalciferol, cholecalciferol, 25-hydroxycholecalciferol, 1-α-25-dihydroxycholecalciferol, or in combination or as supplement with phosphate binders, estrogens, calcitonin or biphosphonates.
